(19) United States
(12) Patent Application Publication
(10) Pub. No.: US 2026/0049346 A1
(43) Pub. Date: Feb. 19, 2026

(54) DETECTION OF NUCLEIC ACIDS

(71) Applicant: MESO SCALE TECHNOLOGIES, LLC., Rockville, MD (US)

(72) Inventors: Annamaria SZABOLCS, Rockville, MD (US); Seth B. HARKINS, Bethesda, MD (US); Timothy J. BREAK, Clarksburg, MD (US)

(21) Appl. No.: 19/297,745

(22) Filed: Aug. 12, 2025

Related U.S. Application Data
(60) Provisional application No. 63/682,497, filed on Aug. 13, 2024.

(51) Int. Cl. C12Q 1/6825; C12Q 1/6806; C12Q 1/6855

(52) U.S. Cl. CPC C12Q 1/6825; C12Q 1/6806; C12Q 1/6855; C12Q 2600/178

(57) ABSTRACT

The present disclosure relates to a method of detecting a nucleic acid analyte, the method comprising contacting an electrode surface comprising a plurality of capture oligonucleotides immobilized on the electrode surface with a solution comprising: a plurality of nucleic acid analytes; and a plurality of detection oligonucleotides, wherein each of the plurality of detection oligonucleotides comprises a first portion and a second portion adjacent to each other, wherein the first portion comprises a sequence that is complementary to a sequence in the capture oligonucleotide and the second portion comprises a sequence that is complementary to a sequence in the nucleic acid analyte.

### BACKGROUND

MicroRNAs (miRNAs) are key regulators of mRNA transcription. Single-Nucleotide Polymorphisms (SNPs) and post-transcriptional modifications of canonical miRNA sequences lead to a wide array of different isoforms of the same miRNA (isomiRNAs/isomiRs). The mRNA targets and effects of these can differ based on their sequence. Changes in the expression of specific isomiRs can be disease predictors or biomarkers (e.g. cancer types). The current methods that are used to detect specific isomiRs and other nucleic acid analytes focus on qPCR-based detection, microarrays, sequencing, mass spectrometry, and hybridization. However, the performance of these methods can be limited by their low throughput, poor reproducibility and sensitivity, high cost, and long and complicated protocols.

### SUMMARY

Described herein are embodiments of assay methods suitable for detection of nucleic acid analytes. In some aspects, the techniques described herein relate to an ECL method of detecting a nucleic acid analyte, the method comprising (a) contacting an electrode surface comprising a plurality of capture oligonucleotides immobilized on the electrode surface with a solution comprising:
a plurality of nucleic acid analytes; and
a plurality of detection oligonucleotides,
wherein each of the plurality of detection oligonucleotides comprises a first portion and a second portion adjacent to each other, wherein the first portion comprises a sequence that is complementary to a sequence in the capture oligonucleotide and the second portion comprises a sequence that is complementary to a sequence in the nucleic acid analyte;
(b) permitting at least one of the plurality of detection oligonucleotides to hybridize to at least one of the plurality of capture oligonucleotides and at least one of the plurality of nucleic acid analytes, thereby forming a complex, comprising the nucleic acid analyte, a detection oligonucleotide and a capture oligonucleotide (“the analyte hybridization complex”);
(c) incubating the analyte hybridization complex from b) under ligation conditions to ligate the nucleic acid analyte to the capture oligonucleotide;
(d) washing the electrode surface to remove any detection oligonucleotides that are not hybridized to the capture oligonucleotide and nucleic acid analyte that are ligated together in c); and
(e) detecting the detection oligonucleotide hybridized to the ligated nucleic acid analyte and capture oligonucleotide following d), said detecting comprising performing an ECL reaction by inducing a charge at the electrode surface in the presence of an ECL detection reagent complexed with the detection oligonucleotide (“the complexed ECL detection reagent-detection oligonucleotide”) and an ECL co-reactant, and measuring any luminescence signal emitted.

### BRIEF DESCRIPTION OF THE DRAWINGS

FIGS. 1A and 1B illustrate embodiments of an ECL assay in the presence and absence of the analyte.

FIG. 2 illustrates an embodiment of a capture oligonucleotide with its 5′ end attached to the surface.

FIG. 3 illustrates an embodiment of complexing using a binding pair, where the first member of the binding pair is biotin attached to the detection oligonucleotide, and the second member of the binding pair is streptavidin (“SA”) attached to the ECL detection reagent.

FIG. 4 illustrates an embodiment of a first portion and a second portion of the detection oligonucleotide.

FIG. 5 illustrates an embodiment of an additional (“further”) portion that is adjacent to the second portion of the detection oligonucleotide.

FIGS. 6A and 6B illustrate an embodiment of a detection oligonucleotide with a stem-loop structure which comprises a hybridization site for a nucleic acid analyte.

FIGS. 7A-7C illustrate an embodiment of an assay when a nucleic acid analyte is ligated to both the capture and detection oligonucleotides, absence of any ligation in the absence of a nucleic acid analyte and the ligation of a nucleic acid analyte to just the capture oligonucleotide.

FIG. 8 illustrates an embodiment of a capture oligonucleotide with its 5′ end attached to the surface which detects only those nucleic acid analytes that are phosphorylated on the 5′ end.

FIG. 9 illustrates an embodiment of a detection oligonucleotide with a stem-loop portion and rolling circle amplification (RCA).

FIG. 10 illustrates an embodiment of an anchor oligonucleotide attached to the electrode surface and hybridized to the concatemer.

FIG. 11 illustrates an embodiment of an electrode surface with a plurality of specific capture oligonucleotides in specific regions of the electrode that bind to specific detection oligonucleotides and specific nucleic acid analytes.

### DETAILED DESCRIPTION

### Methods of Detecting a Nucleic Acid Analyte

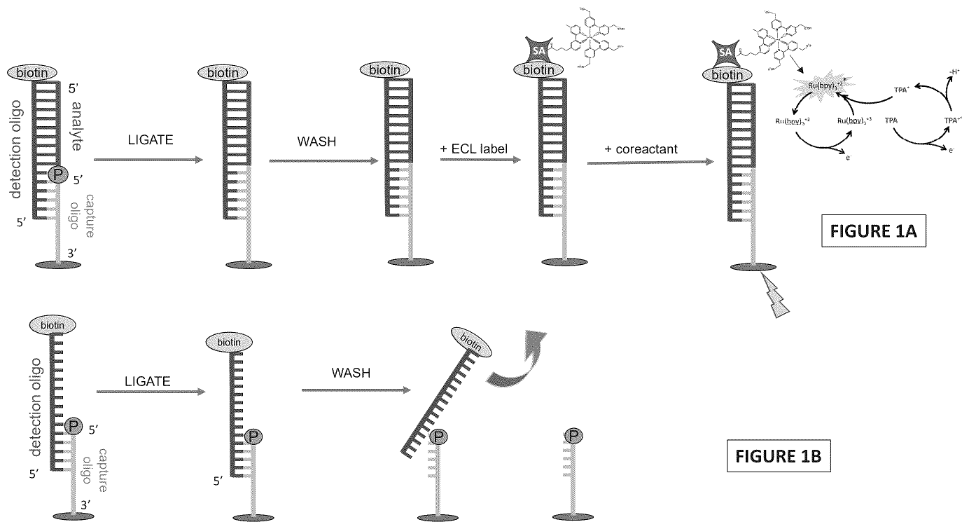

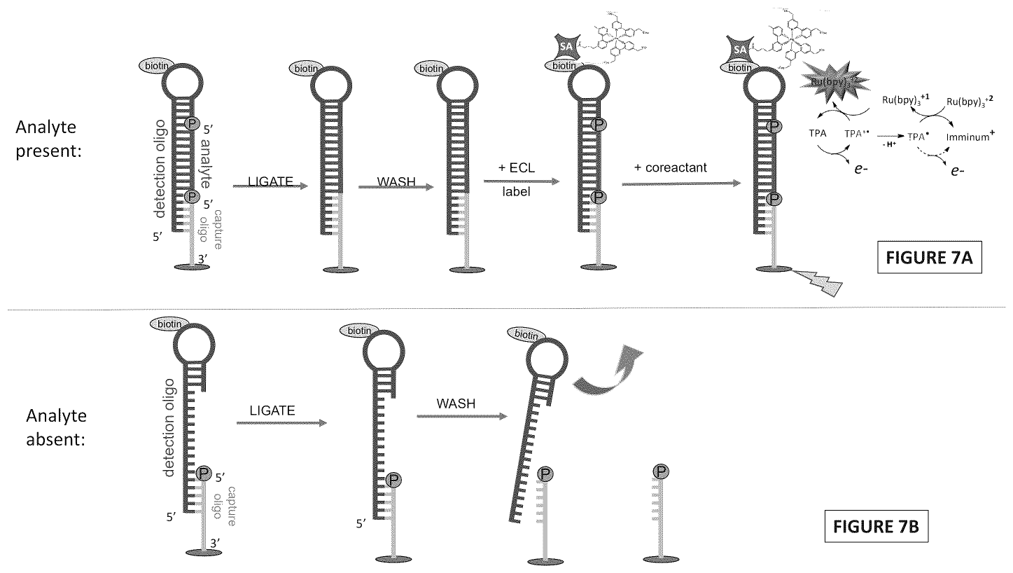

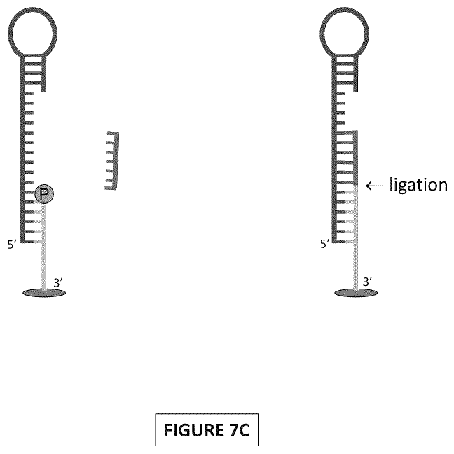

Disclosed herein are embodiments of an ECL method for detecting a nucleic acid analyte (e.g., miRNA, siRNA), for example a nucleic acid analyte in a sample. Disclosed embodiments are methods comprising hybridizing detection oligonucleotides to analytes and capture oligonucleotides (“the analyte hybridization complex”) in a way that the capture oligonucleotide can be ligated to the analyte. The resulting nucleic acid comprising the ligated analyte and capture oligonucleotide provides a more stable hybridization (e.g., higher melting temperature) with the detection oligonucleotide than the hybridization of the detection oligonucleotide to only the capture oligonucleotide (i.e., in the absence of the ligated analyte). This allows for utilizing wash conditions (e.g., temperature and/or salt concentrations) under which detection oligonucleotides which are hybridized to capture oligonucleotides that are not ligated to the analyte will be removed (e.g., as illustrated in the bottom panel of FIGS. 1 and 7), while detection oligonucleotides that are hybridized to the ligated analyte and capture oligonucleotides remain hybridized (upper panel of FIGS. 1 and 7). Following the wash, the remaining hybridized detection oligonucleotides can be detected, for example using an ECL reaction. Thus, the resulting luminescence from the ECL reaction indicates the presence of the analyte nucleic acid, for example in a sample. In some embodiments, the method can differentiate between miRNA or siRNA variants of different length. In some embodiments, the method can be used to detect 3′ modifications and adenylation of miRNA.

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

### Terms

Unless defined otherwise, technical and scientific terms used herein have the same meaning as commonly understood when read in light of the instant disclosure by one of ordinary skill in the art to which the present disclosure belongs. For purposes of the present disclosure, the following terms are explained below.

The disclosure herein uses affirmative language to describe the numerous embodiments. The disclosure also includes embodiments in which subject matter is excluded, in full or in part, such as substances or materials, method steps and conditions, protocols, or procedures.

The articles “a” and “an” are used herein to refer to one or to more than one (for example, at least one) of the grammatical object of the article. By way of example, “an element” means one element or more than one element.

Unless the context clearly indicates otherwise, “about” is meant a quantity, level, value, number, frequency, percentage, dimension, size, amount, weight or length that varies by as much as 10% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The terms “nucleic acid”, “nucleic acid analyte” or “nucleic acid molecule” as used herein have their plain and ordinary meaning as understood in light of the specification, and refer to polynucleotides, such as deoxyribonucleic acid (DNA) or ribonucleic acid (RNA), oligonucleotides, those that appear in a cell naturally, fragments generated by the polymerase chain reaction (PCR), and fragments generated by any of ligation, scission, endonuclease action, and exonuclease action. The term “a plurality of nucleic acid analytes” includes at least one nucleic acid analyte or molecule. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, or phosphoramidate. The term “nucleic acid molecule” also includes so-called “peptide nucleic acids,” which comprise naturally-occurring or modified nucleic acid bases attached to a polyamide backbone. Nucleic acids can be either single stranded or double stranded. “Oligonucleotide” can be used interchangeable with nucleic acid and can refer to either double stranded or single stranded DNA or RNA. A nucleic acid or nucleic acids can be contained in a nucleic acid vector or nucleic acid construct (e.g. plasmid, virus, retrovirus, lentivirus, bacteriophage, cosmid, fosmid, phagemid, bacterial artificial chromosome (BAC), yeast artificial chromosome (YAC), or human artificial chromosome (HAC)) that can be used for amplification and/or expression of the nucleic acid or nucleic acids in various biological systems. Typically, the vector or construct will also contain elements including but not limited to promoters, enhancers, terminators, inducers, ribosome binding sites, translation initiation sites, start codons, stop codons, polyadenylation signals, origins of replication, cloning sites, multiple cloning sites, restriction enzyme sites, epitopes, reporter genes, selection markers, antibiotic selection markers, targeting sequences, peptide purification tags, or accessory genes, or any combination thereof.

The nucleic acids described herein comprise nucleobases. Primary, canonical, natural, or unmodified bases are adenine, cytosine, guanine, thymine, and uracil. Other nucleobases include but are not limited to purines, pyrimidines, modified nucleobases, 5-methylcytosine, pseudouridine, dihydrouridine, inosine, 7-methylguanosine, hypoxanthine, xanthine, 5,6-dihydrouracil, 5-hydroxymethylcytosine, 5-bromouracil, isoguanine, isocytosine, aminoallyl bases, dye-labeled bases, fluorescent bases, or biotin-labeled bases.

The term “capture oligonucleotide” as used herein has its plain and ordinary meaning as understood in light of the specification, and refers to a nucleic acid oligonucleotide that is immobilized on a surface, through chemical linkage or through a binding pair. The capture oligonucleotide has a portion having a sequence which is complementary to a detection oligonucleotide. Methods for immobilizing oligonucleotides on a surface, such as the surface of an electrode used for ECL reactions, are known in the art (see, for example, Balasheb Nimse et al. (2014) Immobilization Techniques for Microarray: Challenges and Applications. Sensors. 14 (2): 22208-22229). Such methods include, but are not limited to one or more of the following mechanisms: (1) physical adsorption, for example, via charge-charge or hydrophobic interactions (2) covalent immobilization, for example, via chemical bonding; and (3) non-covalent protein-ligand interactions such as streptavidin-biotin immobilization. In one aspect, one or more oligonucleotides are immobilized to a functionalized support surface. In one aspect, one or more oligonucleotides are immobilized to a support surface that has not been modified to include one or more functional groups. In one aspect, one or more oligonucleotides are immobilized by physical absorption to a support surface that includes one or more of the following moieties: amine, nitrocellulose, poly(l-lysine), PAAH, and diazonium. In one aspect, one or more oligonucleotides are immobilized to a support surface by covalent interactions, for example through a thiol (—SH), amine (—NH2), or hydrazide group. In one aspect, the support surface includes or is modified to include a reactive functionality, including, for example, carboxyl (—COOH), aldehyde (—CHO), epoxy (—CHCH2O), isothiocyanate (—N═C═S), maleimide (—HC2(CO)2NH), or mercaptosilane (—Si—R—SH). In one aspect, the oligonucleotide includes or is modified to include a reactive functionality, including, for example, a thiol, amine or hydrazide group. In one aspect, one or more oligonucleotides are immobilized to a support surface through a nucleophilic or electrophilic functionality present on the support surface. Oligonucleotide immobilization methods include, but are not limited to, methods employing a protein (for example, streptavidin, avidin, or bovine serum albumin (BSA)) linked or otherwise bound to the oligonucleotide wherein said protein is covalently or non-covalently immobilized on the surface; methods employing Sulfosuccinimidyl-4-(N-maleimidomethyl)cyclohexane-1-carboxylate (Sulfo-SMCC); methods involving incubating the surface (such as a carbon surface) with a solution containing thiol-modified oligonucleotides and allowing the thiol-modified oligonucleotides to irreversibly react with the surface; and methods disclosed in international patent application publication numbers WO2020180645 and WO2014164594, each of which is incorporated herein for its disclosure related to immobilization of oligonucleotides, and in its entirety.

The term “detection oligonucleotide” as used herein has its plain and ordinary meaning as understood in light of the specification, and refers to a nucleic acid oligonucleotide that hybridizes to a capture oligonucleotide and a nucleic acid analyte.

The term “complementary” as used herein in reference to oligonucleotides or portion(s) thereof has its plain and ordinary meaning as understood in light of the specification, and means that the nucleobase sequence (or simply “sequence”) of such oligonucleotide, or referenced portion(s) thereof, matches the nucleobase sequence of another oligonucleotide, or referenced portion(s) thereof, when the two nucleobase sequences are aligned in opposing directions. Nucleobase matches or complementary nucleobases as used herein refer to adenine (A) and thymine (T), adenine (A) and uracil (U), cytosine (C) and guanine (G), and 5-methyl cytosine (mC) and guanine (G), or equivalent pairings known in the art. Complementary oligonucleotides and/or nucleic acids need not have nucleobase complementarity at each nucleoside. Rather, mismatches are permitted. As used herein, “fully complementary” or “100% complementary” means that such oligonucleotides or referenced part(s) thereof are complementary to another oligonucleotide or referenced part(s) thereof at each nucleoside. In such embodiments, mismatches are not permitted.

The term “stem-loop portion” as used herein has its plain and ordinary meaning as understood in light of the specification, and refers to a portion of an oligonucleotide that has a sequence that is self-complementary and which can fold back on itself to form a double stranded portion (the “stem”), the two strands of which are connected by a single-stranded portion (the “loop”). The self-complementary portion can be fully complementary, or only partially complementary (i.e., having one or mismatched nucleobases in the double-stranded portion of the stem-loop structure).

The assay modules (e.g., assay plates or cartridges or multi-well assay plates), methods, ECL detection reagents, co-reactants, ECL reactions, and detection of ECL reaction and apparatuses for conducting assay measurements suitable for the present disclosure are described for example, in US 2004/0022677; US 2005/0052646; US 2005/0142033; US 2004/0189311, US 2022/03735541, US 2022/0018846, US 2022/0018785, US 2023/0175043 and US 2023/0141860, each of which is incorporated herein by reference in their entireties. Assay plates and plate readers are now commercially available (MULTI-SPOT® and MULTI-ARRAY® plates and SECTOR® instruments, MESO SCALE DISCOVERY®).

### Methods of Detecting an Analyte Using Capture Oligonucleotides and Detection Oligonucleotides

In some embodiments, for example as illustrated in FIG. 1, the method comprises:
a) contacting an electrode surface comprising a plurality of capture oligonucleotides (“capture oligo”) that are immobilized on the electrode surface with a solution comprising a plurality of nucleic acid analytes (e.g., miRNA) and a plurality of detection oligonucleotides;
b) permitting at least one of the plurality of detection oligonucleotides to hybridize to at least one of the plurality of capture oligonucleotides and at least one of the plurality of nucleic acid analytes, thereby forming a complex comprising the nucleic acid analyte, a detection oligonucleotide and a capture oligonucleotide (“the analyte hybridization complex”);
c) incubating the analyte hybridization complex from b) under ligation conditions (e.g., in the presence of a ligase and appropriate buffer) to ligate the nucleic acid analyte to the capture oligonucleotide;
d) washing the electrode surface under appropriate wash conditions to leave the detection oligonucleotides hybridized to analyte and capture oligonucleotides that are ligated together, (e.g., FIG. 1, upper panel) but which remove the detection oligonucleotides that are hybridized to the capture oligonucleotides that are not ligated to an analyte oligonucleotide, as well as removing any analyte oligonucleotides that are not ligated to a capture oligonucleotide, (e.g. FIG. 1, lower panel) and
e) detecting the detection oligonucleotides that remain hybridized to the ligated nucleic acid analytes and capture oligonucleotides following the wash in d) by performing an ECL reaction by inducing a charge at the electrode surface in the presence of an ECL detection reagent complexed with the detection oligonucleotide (“the complexed ECL detection reagent-detection oligonucleotide”) and an ECL co-reactant, and measuring any luminescence signal emitted.

Unless expressly stated or otherwise necessarily required, the above items can be carried out in any order. In some embodiments, one or more of the items are combined. For example, in some embodiments, items a)-d), b)-d), a)-c), or b)-c) are performed together and not as discrete steps. In some embodiments, one or more of the items are divided into sub-items. For example, in some embodiments, the electrode surface is first contacted with a solution comprising the plurality of detection oligonucleotides, and then a solution comprising the plurality of nucleic acid analytes is added, thus providing a solution in contact with the electrode surface comprising the plurality of nucleic acid analytes and the plurality of detection oligonucleotides. In some embodiments, the order is reversed, and the nucleic acid analytes are contacted with the electrode surface followed by the detection oligonucleotides. In some embodiments, the nucleic acid analytes are combined in a solution, and the solution comprising both is contacted with the electrode surface. In some embodiments, the hybridization of the detection oligonucleotides to the nucleic acid analytes is performed prior to exposing the detection oligonucleotides and the nucleic acid analytes to the capture oligonucleotides. For example, in some embodiments, the detection oligonucleotides are combined with the nucleic acid analytes and permitted to hybridize, and then a solution comprising the hybridized detection oligonucleotides and nucleic acid analytes are contacted with electrode surface as recited in item a), thereby exposing them to the capture oligonucleotides as recited in item b), and the hybridized detection oligonucleotides and nucleic acid analytes are permitted to hybridize to the capture oligonucleotides thereby forming analyte hybridization complexes as recited in item c). In some embodiments, the hybridization of the detection oligonucleotides to the capture oligonucleotides is performed prior to exposing the detection oligonucleotides and the capture oligonucleotides to the nucleic acid analytes. For example, in some embodiments, the detection oligonucleotides is contacted with the capture oligonucleotides immobilized on the electrode surface and permitted to hybridize, and then a solution comprising the nucleic acid analytes are contacted with the electrode surface as recited in item a), thereby exposing the detection oligonucleotides to the capture oligonucleotides and nucleic acid analytes as recited in item b), and the hybridized detection oligonucleotides and capture oligonucleotides are permitted to hybridize to the nucleic acid analytes thereby forming analyte hybridization complexes as recited in item c). In some embodiments, the hybridization of the detection oligonucleotides to the capture oligonucleotides is performed together with the hybridization of the detection oligonucleotides to the nucleic acid analytes. For example, in some embodiments, a solution comprising the nucleic acid analytes and detection oligonucleotides is contacted with the capture oligonucleotides immobilized on the electrode surface, thereby exposing the detection oligonucleotides to the capture oligonucleotides and the nucleic acid analytes as recited in item b), and the detection oligonucleotides are permitted to hybridize to the capture oligonucleotides and the nucleic acid analytes, thereby forming the complexes as recited in item c).

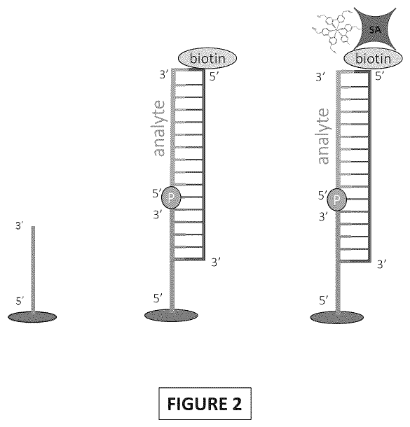

As illustrated in the embodiment depicted in FIG. 1, the capture oligo is oriented with the 3′ end attached to the surface, the detection oligonucleotide hybridizes to the capture oligonucleotide oriented with the 5′ end closest to the surface and the 3′ end furthest from the surface, and the analyte nucleic acid hybridizes with the 3′ end adjacent to the 5′ end of the capture oligonucleotide. As illustrated in FIG. 1, the 5′ end of the capture oligonucleotide is phosphorylated, which allows for ligation to the 3′ end of the nucleic acid analyte. In some embodiments, the 5′ ends of the capture oligonucleotides are subjected to a phosphorylation reaction (e.g., enzyme mediated phosphorylation) prior to or during the ligation (e.g., prior to and/or during item a), b) and/or c), permitting ligation to the 3′ end of the nucleic acid analytes, which will result in hybridization and detection of the detection oligonucleotide. In an alternative embodiment, the orientation of the capture oligonucleotide is inverted from that shown in FIG. 1, instead having the 5′ end of capture oligonucleotide attached to the surface. As a result, the orientation of the hybridized detection oligonucleotide and nucleic acid analyte is likewise inverted from the orientation shown in FIG. 1. An embodiment of this alternate “inverted” configuration is illustrated in FIG. 2. As shown in FIG. 2, the 5′ end of the nucleic acid analyte is phosphorylated, which allows for ligation to the 3′ end of the capture oligonucleotide. In an embodiment, a method using the “inverted” orientation illustrated in the embodiment of FIG. 2 permits detecting only those nucleic acid analytes that are phosphorylated on the 5′ end, because nucleic acid analytes lacking a 5′ phosphate will not be ligated to the capture oligonucleotide, and detection oligonucleotides will not remain hybridized to the un-ligated nucleic acid analytes during the wash (item d)). In an embodiment, a method using the “inverted” orientation illustrated in the embodiment of FIG. 2 can be used to detect phosphorylated nucleic acids of a given sequence but not un-phosphorylated nucleic acids of the same sequence. In an embodiment, the nucleic acid analytes are subjected to a phosphorylation reaction (e.g., enzyme mediated phosphorylation) prior to or during the ligation (e.g., prior to and/or during item a), b) and/or c) so that any nucleic acid analytes lacking 5′ phosphorylation will be phosphorylated, permitting ligation to the 3′ end of the capture oligonucleotide, which will result in hybridization and detection of the detection oligonucleotide.

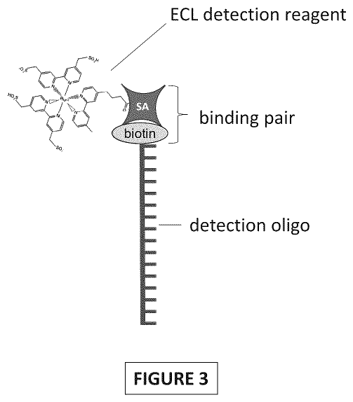

In some embodiments, complexing of the ECL reagent to the detection oligonucleotide (“the complexed ECL detection reagent-detection oligonucleotide”) is by covalent attachment. In some embodiments, the complexing of the ECL reagent to the detection oligonucleotide is accomplished through the binding of a binding pair, where the detection oligonucleotides comprise the first member of the binding pair and the ECL detection reagent comprise the second member of the binding pair. FIG. 3 illustrates an embodiment of complexing using a binding pair, where the first member of the binding pair is biotin attached to the detection oligonucleotide, and the second member of the binding pair is streptavidin (“SA”) attached to the ECL detection reagent, illustrated by the chemical structure attached to the streptavidin. In some embodiments, the ECL reagent can be complexed to the detection oligonucleotide at any point or at multiple points during the methods described herein, but prior to the ECL reaction. In some embodiments, the ECL reagent is complexed to the detection oligonucleotide prior to exposing the detection oligonucleotides to the capture oligonucleotides and the nucleic acid analytes (e.g., prior to item a) or item b)). In some embodiments, the ECL reagent is complexed to the detection oligonucleotide prior to or during hybridization in item b). In some embodiments, the ECL reagent is complexed to the detection oligonucleotide prior to or during the ligation in item c). In some embodiments, the ECL reagent is complexed to the detection oligonucleotide prior to or during the wash in item d). In some embodiments, the ECL reagent is complexed to the detection oligonucleotide after the wash in item d) and prior to performing the ECL reaction of item e). In some embodiments, the binding pair is selected from: antibody/antigen, an oligonucleotide/complementary oligonucleotide, and receptor/ligand, optionally selected from biotin, 2-iminobiotin, avidin, streptavidin, neutravidin, glutathione, glutathione s-transferase, maltose, maltose-binding protein, intein, chitin, chitin-binding protein. In some embodiments, the first and second members of the binding pair are selected from biotin, 2-iminobiotin, avidin, streptavidin, neutravidin. In some embodiments, the binding pair are avidin and streptavidin.

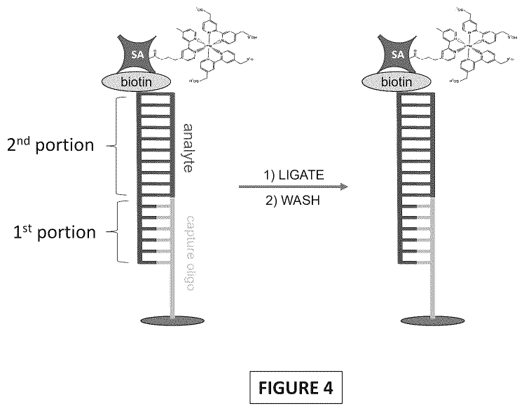

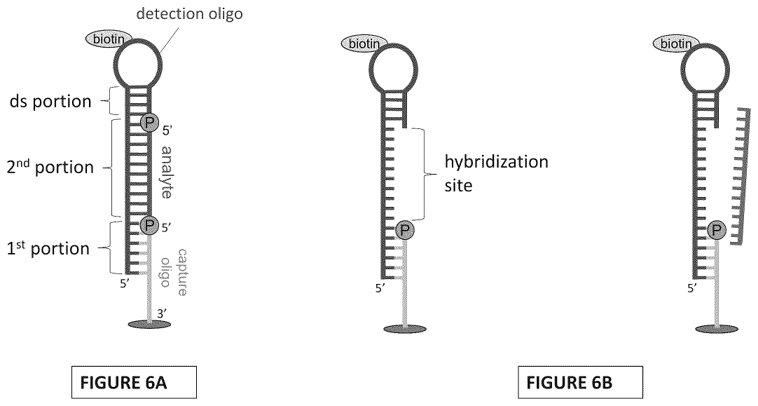

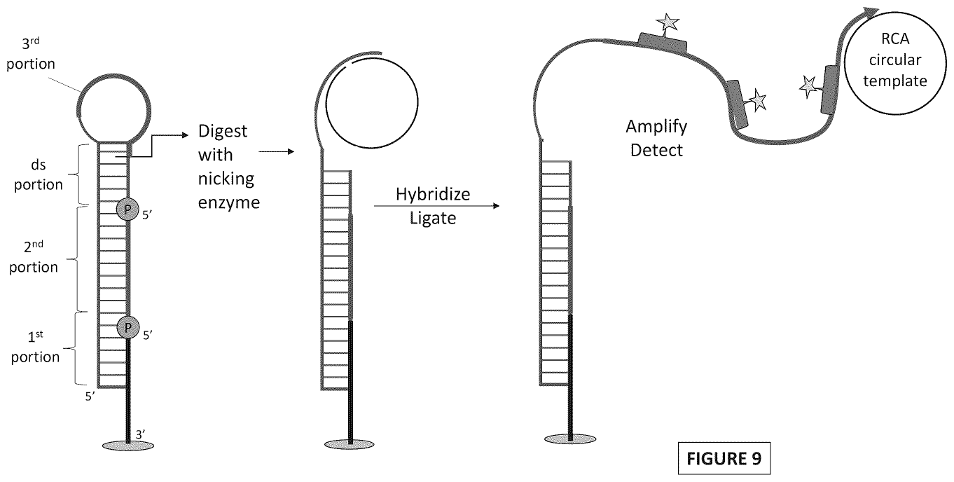

In some embodiments, the length of the detection oligonucleotide is, is about, is at least, or is not more than, 28, 29, 30, 31, 32, 33, 34, 35, 36, 37, 38, 39, 40, 41, 42, 43, 44, 45, 46, 47, 48, 49, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110 or 120 nucleotides long, or a range defined by any two of the aforementioned values, for example, 28-120, 27-49, 25-50, 28-100, 35-60, 40-75, 50-95, 61-91, 60-100, 75-110, 76-106 or 75-120 nucleotides long. In some embodiments, the detection oligonucleotide is a linear probe as illustrated in FIG. 1, wherein the length of the probe is 32 nucleotides long or about 32 nucleotides long. In some embodiments, the detection oligonucleotide is a loop probe as illustrated in FIG. 6, wherein the length of the probe is 76 nucleotides long or about 76 nucleotides long. In some embodiments, the detection oligonucleotide is a turbo-boost loop probe as illustrated in FIG. 9, wherein the length of the probe is 91 nucleotides long or about 91 nucleotides long. In some embodiments, the detection oligonucleotide comprises a first portion and a second portion adjacent to each other, for example as illustrated in FIG. 4, wherein the first portion comprises a sequence that is complementary to a sequence in the capture oligonucleotide (“capture oligo”) and the second portion comprises a sequence that is complementary to a sequence in the nucleic acid analyte (e.g., miRNA). In some embodiments, the length of the complementary portion between the first portion of the detection oligonucleotide and the capture oligonucleotide is, is about, is at least, or is not more than, 8, 9, 10, 11, 12, 13, 14, 15, 16, 17, 18, 19, 20, 21, 22, 23, 24, 25, 30, 31, 32, 33, 34, 35, 36, 37, 38, 39 or 40 nucleotides long, or a range defined by any two of the aforementioned values, for example, 8-40, 10-18, 15-25, or 20-40 nucleotides long. In some embodiments, the length of the complementary portion between the detection oligonucleotide and the capture oligonucleotide is 12 nucleotides long or about 12 nucleotides long. In some embodiments, the complementary portion between the detection oligonucleotide and the capture oligonucleotide is fully complementary as measured over the entirety of the portion. In some embodiments, the complementary portion between the detection oligonucleotide and the capture oligonucleotide as measured over the entirety of the portion is not fully complementary; optionally it is, is about, or is at least, 85%, 86%, 87%, 88%, 89%, 90%, 91%, 92%, 93%, 94%, 95%, 96%, 97%, 98%, or 99% complementary. In some embodiments, the length of the complementary portion between the second portion of the detection oligonucleotide and the nucleic acid analyte is, is about, is at least, or is not more than, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides long, or a range defined by any two of the aforementioned values, for example, 5-35, 15-35, or 5-25, nucleotides long. In some embodiments, the length of the complementary portion between the second portion of the detection oligonucleotide and the nucleic acid analyte is, is about, is at least, or is not more than, 21, 22, 23, 24, or 25 nucleotides long, or a range defined by any two of the aforementioned values, for example, 21-23, or 21-25 nucleotides long. In some embodiments, the length of the complementary portion between the second portion of the detection oligonucleotide and the nucleic acid analyte is 21-23 nucleotides long. In some embodiments, the length of the complementary portion between the second portion of the detection oligonucleotide and the nucleic acid analyte is 21-25 nucleotides long. In some embodiments, the length of the second portion of the detection oligonucleotide is, is about, is at least, or is not more than, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides long, or a range defined by any two of the aforementioned values, for example, 5-35, 15-35, or 5-25, nucleotides long. In some embodiments, the length of the second portion of the detection oligonucleotide is, is about, is at least, or is not more than, 21, 22, 23, 24, or 25 nucleotides long, or a range defined by any two of the aforementioned values, for example, 21-23, or 21-25 nucleotides long. In some embodiments, the length of the second portion of the detection oligonucleotide is 21-23 nucleotides long. In some embodiments, the length of the second portion of the detection oligonucleotide is 21-25 nucleotides long. In some embodiments, the second portion of the detection oligonucleotide is shorter than the length of the analyte. In other embodiments, the second portion of the detection oligonucleotide is longer than the length of the analyte. In some embodiments, the length of the second portion of the detection oligonucleotide is 0, 1, 2, 3, 4, or 5 nucleotides, or a range defined by any two of the preceding values, for example 0-5, 1-4, 0-3, 2-4 or 3-5 nucleotides, shorter or longer than the length or the nucleic acid analyte. In some embodiments, the second portion of the detection oligonucleotide is of the same length as of the analyte. In some embodiments, the nucleic acid analyte is, is about, is at least, or is not more than 16, 17, 18, 19, 20, 21, 22, 23, 24, 25, 26, 27, 28, 29, 30, 31, 32, 33, 34, or 35 nucleotides long, or a range defined by any two of the aforementioned values, for example, 16-35, 20-30, 17-22, 21-25 or 22-34 nucleotides long. In some embodiments, the nucleic acid analyte is 21, 22, 23, 24, or 25 nucleotides long, or a range defined by any two of the aforementioned values, for example, 21-23, or 21-25 nucleotides long. In some embodiments, the nucleic acid analyte is 21-23 nucleotides long. In some embodiments, the nucleic acid analyte is 21-25 nucleotides long. In some embodiments, the complementary portion between the detection oligonucleotide and the nucleic acid analyte is fully complementary as measured over the entirety of the portion, optionally as measured over the entirety of the nucleic acid analyte. In some embodiments, the complementary portion between the detection oligonucleotide and the nucleic acid analyte as measured over the entirety of the portion, optionally as measured over the entirety of the nucleic acid analyte, is not fully complementary; optionally it is, is about, or is at least, 85%, 86%, 87%, 88%, 89%, 90%, 91%, 92%, 93%, 94%, 95%, 96%, 97%, 98%, or 99% complementary. In some embodiments, the nucleic acid analyte is a microRNA (miRNA). In some embodiments, the nucleic acid analyte is a small interfering RNA (siRNA). In some embodiments, the detection oligonucleotides is 27-110 nucleotides long, the first portion is 8-24 nucleotides long, the first portion is at least 80% complementary to the capture oligonucleotide as measured over the entire length of the first portion, the second portion is 21-25 nucleotides long, the nucleic acid analyte is 21-25 nucleotides long, and the second portion is at least 80% complementary to the nucleic acid analyte as measured over the entire length of the nucleic acid analyte. In some embodiments, the nucleic acid analyte is a small interfering RNA (siRNA). In some embodiments, the detection oligonucleotides is 27-110 nucleotides long, the first portion is 8-24 nucleotides long, the first portion is at least 98% complementary to the capture oligonucleotide as measured over the entire length of the first portion, the second portion is 21-25 nucleotides long, the nucleic acid analyte is 21-25 nucleotides long, and the second portion is at least 98% complementary to the nucleic acid analyte as measured over the entire length of the nucleic acid analyte.

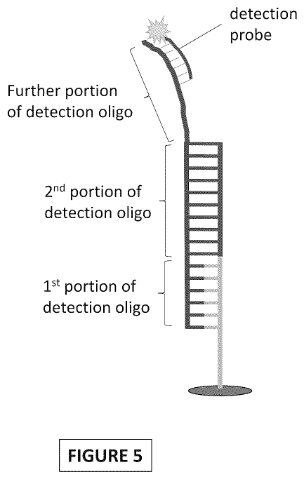

In some embodiments, the detection oligonucleotides comprise an additional (“further”) portion that is adjacent to the second portion as illustrated in FIG. 5, wherein the additional portion comprises a detection sequence that is complementary to a sequence in a detection probe. In some embodiments, the detection includes exposing the detection oligonucleotides hybridized to the ligated nucleic acid analytes and capture oligonucleotides to the detection probes; permitting the detection probes to hybridize to the detection oligonucleotides; and then conducting the ECL reaction, where the detection probe is labeled with the ECL detection reagent. In this way the detection probe hybridizing to the detection oligonucleotide complexes the ECL detection reagent with the detection oligonucleotide. In some embodiments, the method comprises a wash to remove unhybridized detection probes prior to conducting the ECL reaction.

In some embodiments where the detection oligonucleotides comprise an additional portion complementary to the detection probe as described above, and elsewhere herein, the labeling of the detection probe with the ECL detection reagent is by covalent attachment. In some embodiments, the labeling of the detection probe with the ECL detection reagent is through the binding of a binding pair, where the detection probe comprises the first member of the binding pair and the ECL detection reagents comprise the second member of the binding pair. In some embodiments, the detection probe is labeled with the ECL detection reagent prior to hybridization with the detection oligonucleotide. In other embodiments the detection probe is labeled with the ECL detection reagent after it is hybridized to the detection oligonucleotide. In some embodiments, the binding pair is selected from: antibody/antigen, an oligonucleotide/complementary oligonucleotide, and receptor/ligand, optionally selected from biotin, 2-iminobiotin, avidin, streptavidin, neutravidin, glutathione, glutathione s-transferase, maltose, maltose-binding protein, intein, chitin, chitin-binding protein. In some embodiments, the first and second members of the binding pair are selected from biotin, 2-iminobiotin, avidin, streptavidin, neutravidin. In some embodiments, the binding pair are avidin and streptavidin. In some embodiments, the length of the complementary portion between the detection sequence of the detection oligonucleotide and the detection probe is, is about, is at least, or is not more than, 8, 9, 10, 11, 12, 13, 14, 15, 16, 17, 18, 19, 20, 21, 22, 23, 24, 25, 30, 31, 32, 33, 34, 35, 36, 37, 38, 39 or 40 nucleotides long, or a range defined by any two of the aforementioned values, for example, 8-40, 8-25, 10-18, 15-25, or 20-40 nucleotides long. In some embodiments, the length of the complementary portion between the detection sequence of the detection oligonucleotide and the detection probe is 20 nucleotides long or about 20 nucleotides long. In some embodiments, the length of the detection probe and/or the detection sequence is, is about, is at least, or is not more than, 8, 9, 10, 11, 12, 13, 14, 15, 16, 17, 18, 19, 20, 21, 22, 23, 24, 25, 30, 31, 32, 33, 34, 35, 36, 37, 38, 39 or 40 nucleotides long, or a range defined by any two of the aforementioned values, for example, 8-40, 8-25, 10-18, 15-25, or 20-40 nucleotides long. In some embodiments, the detection probe and/or the detection sequence is 20 nucleotides long or about 20 nucleotides long. In some embodiments, the complementary portion between the detection sequence of the detection oligonucleotide and the detection probe is fully complementary as measured over the entirety of the portion, optionally as measured over the entirety of the detection probe and/or detection sequence. In some embodiments, the complementary portion between the detection sequence of the detection oligonucleotide and the detection probe as measured over the entirety of the portion, optionally as measured over the entirety of the detection probe and/or detection sequence, is not fully complementary; optionally it is, is about, or is at least, 85%, 86%, 87%, 88%, 89%, 90%, 91%, 92%, 93%, 94%, 95%, 96%, 97%, 98%, or 99% complementary. In some embodiments, the detection oligonucleotides are, are about 27-49, 61-91, or 76-106 nucleotides long, the first portion is 8-24 nucleotides long, the first portion is at least 80% complementary to the capture oligonucleotide as measured over the entire length of the first portion, the second portion is 21-25 nucleotides long, the nucleic acid analyte is 21-25 nucleotides long, the second portion is at least 80% complementary to the nucleic acid analyte as measured over the entire length of the nucleic acid analyte, the detection sequence and the detection probe are each 8-25 long, and the detection sequence is at least 80% complementary to the detection probe as measured over the entire length of the detection sequence and/or detection probe. In some embodiments, the detection oligonucleotides is, is about 32, 76, or 91 nucleotides long, the first portion is 8-24 nucleotides long, the first portion is at least 80% complementary to the capture oligonucleotide as measured over the entire length of the first portion, the second portion is 21-25 nucleotides long, the nucleic acid analyte is 21-25 nucleotides long, the second portion is at least 80% complementary to the nucleic acid analyte as measured over the entire length of the nucleic acid analyte, the detection sequence and the detection probe are each 8-25 long, and the detection sequence is at least 80% complementary to the detection probe as measured over the entire length of the detection sequence and/or detection probe. In some embodiments, the detection oligonucleotides is, is about 32, 76, or 91 nucleotides long, the first portion is 8-24 nucleotides long, the first portion is at least 98% complementary to the capture oligonucleotide as measured over the entire length of the first portion, the second portion is 21-25 nucleotides long, the nucleic acid analyte is 21-25 nucleotides long, the second portion is at least 98% complementary to the nucleic acid analyte as measured over the entire length of the nucleic acid analyte, the detection sequence and the detection probe are each 8-25 long, and the detection sequence is at least 98% complementary to the detection probe as measured over the entire length of the detection sequence and/or detection probe.

Methods of Detecting an Analyte Using Detection Oligonucleotides with a Stem-Loop Portion

In embodiments of the methods described herein the detection oligonucleotide has a stem-loop structure. In some embodiments, the detection oligonucleotide has a stem-loop structure, an embodiment of which is illustrated in FIG. 6A. As illustrated in the embodiment in FIG. 6, the stem portion of the detection oligonucleotide comprises a single-stranded portion and a double-stranded portion (“ds portion”) when the stem portion is closed (the double-stranded portion also includes the single-stranded loop when the stem portion is closed), where the single-stranded portion comprises a first portion that comprises a sequence that is complementary to the sequence in the capture oligonucleotide and a second portion that comprises a sequence that is complementary to the sequence in the nucleic acid analyte. As illustrated in the embodiment in FIG. 6A, the double-stranded portion and the first portion flank the second portion. In some embodiments, when the stem-loop detection oligonucleotide is hybridized to the capture oligonucleotide, the stem-loop structure provides a hybridization site for the analyte nucleic acid that is of a limited size, blocking the complete hybridization of an analyte nucleic acid that is larger than the hybridization site. In the embodiment illustrated in FIG. 6A, the double-stranded portion (“ds portion”) and the capture oligonucleotide (“capture oligo”) define the hybridization site on the detection oligonucleotide (the portion of the detection oligonucleotide that is single stranded prior to the hybridization of the analyte nucleic acid) that in the depicted embodiment is exactly the same size as the nucleic acid analyte (“miRNA”). A nucleic acid analyte larger than the hybridization site would not be able to completely hybridize to the detection oligonucleotide as there would be a portion on the 3′ and/or 5′ end of the analyte which overhangs the hybridization site, as illustrated in FIG. 6B. As discussed above, and elsewhere herein, embodiments of the method comprise item b) permitting the detection oligonucleotides to hybridize to the capture oligonucleotides and the nucleic acid analytes, thereby forming a plurality of complexes (“the analyte hybridization complexes”) where each analyte hybridization complex comprises a nucleic acid analyte, a detection oligonucleotide and a capture oligonucleotide; d) incubating the plurality of complexes from item c) under ligation conditions (e.g., in the presence of a ligase and appropriate buffer) to ligate the nucleic acid analytes to the capture oligonucleotides; and d) washing the electrode surface under appropriate wash conditions to leave the detection oligonucleotides hybridized to analyte and capture oligonucleotides that are ligated together, (e.g., FIG. 7A) but which remove the detection oligonucleotides that are hybridized to the capture oligonucleotides that are not ligated to an analyte oligonucleotide, as well as removing any analyte oligonucleotides that are not ligated to a capture oligonucleotide, (e.g. FIG. 7B). The embodiment illustrated in FIG. 7A illustrates ligation of the nucleic acid analyte to the capture oligonucleotide and the stem portion of the detection oligonucleotide. In some embodiments, ligation occurs at only between the nucleic acid analyte and the capture oligonucleotide, and the nucleic acid analyte is not ligated to the detection oligonucleotide (e.g., FIG. 7C). In some embodiments, this single ligation is sufficient to permit selective washing of detection oligonucleotides that are hybridized to capture oligonucleotides that have not been ligated to a nucleic acid analyte. As discussed above, and elsewhere herein, following washing, the method comprises: e) detecting the detection oligonucleotides that: i) are ligated to the nucleic acid analyte which is ligated to the capture oligonucleotide (e.g., FIG. 7A), and/or ii) remain hybridized to the ligated nucleic acid analytes and capture oligonucleotides, following the wash in item d), by performing an ECL reaction by inducing a charge at the electrode surface in the presence of an ECL detection reagent complexed with the detection oligonucleotide (“the complexed ECL detection reagent-detection oligonucleotide”) and an ECL co-reactant, and measuring any luminescence signal emitted.

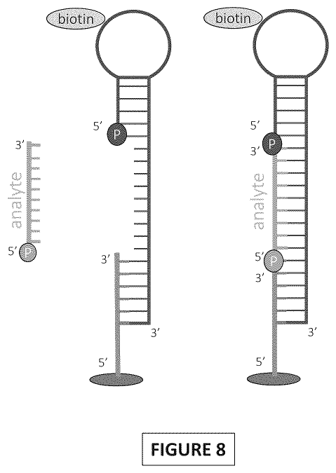

As illustrated in the embodiment depicted in FIG. 7, the capture oligo is oriented with the 3′ end attached to the surface, the detection oligonucleotide hybridizes to the capture oligonucleotide oriented with the 5′ end closest to the surface and the 3′ end furthest from the surface, and the analyte nucleic acid hybridizes with the 3′ end adjacent to the 5′ end of the capture oligonucleotide. As illustrated in FIG. 7, the 5′ end of the capture oligonucleotide is phosphorylated, which allows for ligation to the 3′ end of the nucleic acid analyte. In some embodiments, the 5′ ends of the capture oligonucleotides are subjected to a phosphorylation reaction (e.g., enzyme mediated phosphorylation) prior to or during the ligation (e.g., prior to and/or during item a), b) and/or c), permitting ligation to the 3′ end of the nucleic acid analytes, which will result in hybridization and detection of the detection oligonucleotide. In addition, in some embodiments, the 5′ end of the nucleic acid analyte can be phosphorylated, which allows for ligation to the 3′ end of the detection oligonucleotide. In an alternative embodiment, the orientation of the capture oligonucleotide is inverted from that shown in FIG. 7, instead having the 5′ end of capture oligonucleotide attached to the surface. As a result, the orientation of the hybridized detection oligonucleotide and nucleic acid analyte is likewise inverted from the orientation shown in FIG. 7. An embodiment of this alternate “inverted” configuration is illustrated in FIG. 8. As shown in FIG. 8, the 5′ end of the nucleic acid analyte is phosphorylated, which allows for ligation to the 3′ end of the capture oligonucleotide. In some embodiments, the 5′ end of the detection oligonucleotide is phosphorylated, which allows for ligation to the 3′ end of the nucleic acid analyte, as shown in the embodiment illustrated in FIG. 8. In an embodiment, a method using the “inverted” orientation illustrated in the embodiment of FIG. 8 permits detecting only those nucleic acid analytes that are phosphorylated on the 5′ end, because nucleic acid analytes lacking a 5′ phosphate will not be ligated to the capture oligonucleotide, and detection oligonucleotides will not remain hybridized to the un-ligated nucleic acid analytes during the wash (item d)), even if they nucleic acid analyte is ligated to the detection oligonucleotide. In an embodiment, a method using the “inverted” orientation illustrated in the embodiment of FIG. 8 can be used to detect phosphorylated nucleic acids of a given sequence but not un-phosphorylated nucleic acids of the same sequence. In an embodiment, the nucleic acid analytes are subjected to a phosphorylation reaction (e.g., enzyme mediated phosphorylation) prior to or during the ligation (e.g., prior to and/or during item a), b) and/or c) so that any nucleic acid analytes lacking 5′ phosphorylation will be phosphorylated, permitting ligation to the 3′ end of the capture oligonucleotide, which will result in hybridization and detection of the detection oligonucleotide.

As discussed above, and elsewhere herein, in some embodiments, complexing of the ECL reagent to the detection oligonucleotide (“the complexed ECL detection reagent-detection oligonucleotide”) is by covalent attachment. As discussed above, and elsewhere herein, in some embodiments, the complexing of the ECL reagent to the detection oligonucleotide is accomplished through the binding of a binding pair, where the detection oligonucleotides comprise the first member of the binding pair and the ECL detection reagent comprise the second member of the binding pair. FIG. 7A illustrates an embodiment of complexing using a binding pair, where the first member of the binding pair is biotin attached to the detection oligonucleotide, and the second member of the binding pair is streptavidin attached to the ECL detection reagent (s. In some embodiments, the first member of the binding pair is located on the stem portion and/or the loop portion of the detection oligonucleotide. In some embodiments, the ECL reagent can be complexed to the detection oligonucleotide at any point or at multiple points during the methods described herein, but prior to the ECL reaction. In some embodiments, the ECL reagent is complexed to the detection oligonucleotide prior to exposing the detection oligonucleotides to the capture oligonucleotides and the nucleic acid analytes (e.g., prior to item a) or item b)). In some embodiments, the ECL reagent is complexed to the detection oligonucleotide prior to or during hybridization in item b). In some embodiments, the ECL reagent is complexed to the detection oligonucleotide prior to or during the ligation in item c). In some embodiments, the ECL reagent is complexed to the detection oligonucleotide prior to or during the wash in item d). In some embodiments, the ECL reagent is complexed to the detection oligonucleotide after the wash in item d) and prior to performing the ECL reaction of item e). In some embodiments, the binding pair is selected from: antibody/antigen, an oligonucleotide/complementary oligonucleotide, and receptor/ligand, optionally selected from biotin, 2-iminobiotin, avidin, streptavidin, neutravidin, glutathione, glutathione s-transferase, maltose, maltose-binding protein, intein, chitin, chitin-binding protein. In some embodiments, the first and second members of the binding pair are selected from biotin, 2-iminobiotin, avidin, streptavidin, neutravidin. In some embodiments, the binding pair are avidin and streptavidin.

In some embodiments, the length of the detection oligonucleotide comprising a stem-loop portion is, is about, is at least, or is not more than, 28, 29, 30, 31, 32, 33, 34, 35, 36, 37, 38, 39, 40, 41, 42, 43, 44, 45, 46, 47, 48, 49, 50, 55, 60, 61, 62, 63, 64, 65, 66, 67, 68, 69, 70, 75, 80, 81, 82, 83, 84, 85, 86, 87, 88, 89, 90, 91, 92, 93, 94, 95, 96, 97, 98, 99, or 100 nucleotides long, or a range defined by any two of the aforementioned values, for example, 28-100, 35-60, 40-75, 61-91, 50-95, or 80-100 nucleotides long. In some embodiments, the length of the detection oligonucleotide comprising a stem-loop portion is 76 nucleotides long or about 76 nucleotides long. In some embodiments, the double-stranded stem portion of the detection oligonucleotide is, is about, is at least, or is not more than, 5, 6, 7, 8, 9, 10, 11, 12, 13, 14, 15, 16, 17, 18, 19, 20, 21, 22, 23, 24, 25 or 30 nucleotides long, or a range defined by any two of the aforementioned values, for example, 5-30, 5-20, 10-25 or 15-30 nucleotides long. In some embodiments, the length of the double-stranded stem portion is 14 nucleotides long or about 14 nucleotides long. In some embodiments, the length of the double-stranded stem portion is 20 nucleotides long or about 20 nucleotides long. In some embodiments, loop portion of the detection oligonucleotide is, is about, is at least, or is not more than, 3, 4, 5, 6, 7, 8, 9, 10, 11, 12, 13, 14, 15, 16, 17, 18, 19 or 20 nucleotides long, or a range defined by any two of the aforementioned values, for example, 3-20, 3-15, 5-15 or 5-10 nucleotides long. In some embodiments, the length of the loop portion is 16 nucleotides long or about 16 nucleotides long. In some embodiments, the length of the loop portion is 20 nucleotides long or about 20 nucleotides long. In some embodiments, detection oligonucleotide comprises a first portion and a second portion adjacent to each other, for example as illustrated in FIG. 6, wherein the first portion comprises a sequence that is complementary to a sequence in the capture oligonucleotide (“capture oligo”) and the second portion comprises a sequence that is complementary to a sequence in the nucleic acid analyte (e.g., miRNA). In some embodiments, the length of the complementary portion between the first portion of the detection oligonucleotide comprising a stem-loop portion and the capture oligonucleotide is, is about, is at least, or is not more than, 8, 9, 10, 11, 12, 13, 14, 15, 16, 17, 18, 19, 20, 21, 22, 23, 24, 25, 30, 31, 32, 33, 34, 35, 36, 37, 38, 39 or 40 nucleotides long, or a range defined by any two of the aforementioned values, for example, 8-40, 10-18, 15-25, or 20-40 nucleotides long. In some embodiments, the length of the complementary portion between the detection oligonucleotide comprising a stem-loop portion and the capture oligonucleotide is 12 nucleotides long or about 12 nucleotides long. In some embodiments, the complementary portion between the detection oligonucleotide and the capture oligonucleotide is fully complementary as measured over the entirety of the portion. In some embodiments, the complementary portion between the detection oligonucleotide and the capture oligonucleotide as measured over the entirety of the portion is not fully complementary; optionally it is, is about, or is at least, 85%, 86%, 87%, 88%, 89%, 90%, 91%, 92%, 93%, 94%, 95%, 96%, 97%, 98%, or 99% complementary. In some embodiments, the length of the complementary portion between the second portion of the detection oligonucleotide comprising a stem-loop portion and the nucleic acid analyte is, is about, is at least, or is not more than, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides long, or a range defined by any two of the aforementioned values, for example, 5-35, 15-35, or 5-25, nucleotides long. In some embodiments, the length of the complementary portion between the second portion of the detection oligonucleotide comprising a stem-loop portion and the nucleic acid analyte is, is about, is at least, or is not more than, 21, 22, 23, 24, or 25 nucleotides long, or a range defined by any two of the aforementioned values, for example, 21-23, or 21-25 nucleotides long. In some embodiments, the length of the complementary portion between the second portion of the detection oligonucleotide comprising a stem-loop portion and the nucleic acid analyte is 21-23 nucleotides long. In some embodiments, the length of the complementary portion between the second portion of the detection oligonucleotide comprising a stem-loop portion and the nucleic acid analyte is 21-25 nucleotides long. In some embodiments, the length of the second portion of the detection oligonucleotide comprising a stem-loop portion is, is about, is at least, or is not more than, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides long, or a range defined by any two of the aforementioned values, for example, 5-35, 15-35, or 5-25, nucleotides long. In some embodiments, the length of the second portion of the detection oligonucleotide comprising a stem-loop portion is, is about, is at least, or is not more than, 21, 22, 23, 24, or 25 nucleotides long, or a range defined by any two of the aforementioned values, for example, 21-23, or 21-25 nucleotides long. In some embodiments, the length of the second portion of the detection oligonucleotide comprising a stem-loop portion is 21-23 nucleotides long. In some embodiments, the length of the second portion of the detection oligonucleotide comprising a stem-loop portion is 21-25 nucleotides long. In some embodiments, the second portion of the detection oligonucleotide comprising a stem-loop portion is shorter than the length of the analyte. In other embodiments, the second portion of the detection oligonucleotide comprising a stem-loop portion is longer than the length of the analyte. In some embodiments, the length of the second portion of the detection oligonucleotide is 0, 1, 2, 3, 4, or 5 nucleotides, or a range defined by any two of the preceding values, for example 0-5, 1-4, 0-3, 2-4 or 3-5 nucleotides, shorter or longer than the length or the nucleic acid analyte. In some embodiments, the second portion of the detection oligonucleotide comprising a stem-loop portion is of the same length as of the analyte. In some embodiments, the nucleic acid analyte is, is about, is at least, or is not more than 16, 17, 18, 19, 20, 21, 22, 23, 24, 25, 26, 27, 28, 29, 30, 31, 32, 33, 34, or 35 nucleotides long, or a range defined by any two of the aforementioned values, for example, 16-35, 20-30, 17-22, 21-25 or 22-34 nucleotides long. In some embodiments, the nucleic acid analyte is 21, 22, 23, 24, or 25 nucleotides long, or a range defined by any two of the aforementioned values, for example, 21-23, or 21-25 nucleotides long. In some embodiments, the nucleic acid analyte is 21-23 nucleotides long. In some embodiments, the nucleic acid analyte is 21-25 nucleotides long. In some embodiments, the complementary portion between the detection oligonucleotide and the nucleic acid analyte is fully complementary as measured over the entirety of the portion, optionally as measured over the entirety of the nucleic acid analyte. In some embodiments, the complementary portion between the detection oligonucleotide and the nucleic acid analyte as measured over the entirety of the portion, optionally as measured over the entirety of the nucleic acid analyte, is not fully complementary; optionally it is, is about, or is at least, 85%, 86%, 87%, 88%, 89%, 90%, 91%, 92%, 93%, 94%, 95%, 96%, 97%, 98%, or 99% complementary. In some embodiments, the nucleic acid analyte is a microRNA (miRNA). In some embodiments, the nucleic acid analyte is a small interfering RNA (siRNA). In some embodiments, the detection oligonucleotides comprising a stem-loop portions is 61-106 nucleotides long, the stem-loop portion is 8-30 nucleotides long, the first portion is 8-24 nucleotides long, the first portion is at least 80% complementary to the capture oligonucleotide as measured over the entire length of the first portion, the second portion is 21-25 nucleotides long, the nucleic acid analyte is 21-25 nucleotides long, and the second portion is at least 80% complementary to the nucleic acid analyte as measured over the entire length of the nucleic acid analyte. In some embodiments, the detection oligonucleotides comprising a stem-loop portions is 61-106 nucleotides long, the stem-loop portion is 8-30 nucleotides long, the first portion is 8-24 nucleotides long, the first portion is at least 98% complementary to the capture oligonucleotide as measured over the entire length of the first portion, the second portion is 21-25 nucleotides long, the nucleic acid analyte is 21-25 nucleotides long, and the second portion is at least 98% complementary to the nucleic acid analyte as measured over the entire length of the nucleic acid analyte.

In some embodiments, the detection oligonucleotides comprising a stem-loop portion comprise an additional (“further”) portion that is adjacent to the second portion as illustrated in the embodiment depicted in FIG. 5, wherein the additional portion comprises a detection sequence that is complementary to a sequence in a detection probe. In some embodiments, the detection includes exposing the detection oligonucleotides comprising a stem-loop portions hybridized to the ligated nucleic acid analytes and capture oligonucleotides to the detection probes; permitting the detection probes to hybridize to the detection oligonucleotides at the detection sequence; and then conducting the ECL reaction, where the detection probe is labeled with the ECL detection reagent. In this way the detection probe hybridizing to the detection oligonucleotide complexes the ECL detection reagent with the detection oligonucleotide (“the complexed ECL detection reagent-detection oligonucleotide”) comprising a stem-loop portion. In some embodiments, the method comprises a wash to remove unhybridized detection probes prior to conducting the ECL reaction. In some embodiments, this detection sequence is within the stem-loop portion of the detection oligonucleotide. In some embodiments, the method comprises opening the stem-loop portion of the detection oligonucleotide so that the detection probe has access to, or better access to, the additional portion of the detection probe to which it hybridizes. In some embodiments, the opening of the stem-loop comprises exposing the detection oligonucleotide to conditions that disrupt hybridization in the stem-loop (e.g., temperature and/or salt concentrations). In some embodiments, the opening of the stem-loop comprises cutting the nucleic acid strand (e.g., using an enzyme to cut or “nick” the nucleic acid) in the loop portion or stem portion such that the loop opens. In some embodiments, the detecting in item e) comprises: i) exposing the detection oligonucleotides having a stem-loop portions that are hybridized to the ligated nucleic acid analytes and capture oligonucleotides to a nicking enzyme that cleaves one strand of detection oligonucleotides, thereby nicking the detection oligonucleotides; ii) exposing the nicked detection oligonucleotides of i) to detection probes comprising a sequence complementary to the detection sequence; permitting hybridization of the detection probes to the nicked detection oligonucleotides; and iii) conducting the ECL reaction, optionally where a wash to remove unhybridized detection probes is conducted prior to conducting the ECL reaction.

In some embodiments wherein the detection probes comprise a stem-loop portion comprising an additional portion complementary to a detection probe as discussed above, and elsewhere herein, the labeling of the detection probe with the ECL detection reagent is by covalent attachment. In some embodiments, the labeling of the detection probe with the ECL detection reagent is through the binding of a binding pair, where the detection probe comprises the first member of the binding pair and the ECL detection reagents comprise the second member of the binding pair. In some embodiments, the detection probe is labeled with the ECL detection reagent prior to hybridization with the detection oligonucleotide comprising a stem-loop portion. In other embodiments the detection probe is labeled with the ECL detection reagent after it is hybridized to the detection oligonucleotide comprising a stem-loop portion. In some embodiments, the binding pair is selected from: antibody/antigen, an oligonucleotide/complementary oligonucleotide, and receptor/ligand, optionally selected from biotin, 2-iminobiotin, avidin, streptavidin, neutravidin, glutathione, glutathione s-transferase, maltose, maltose-binding protein, intein, chitin, chitin-binding protein. In some embodiments, the first and second members of the binding pair are selected from biotin, 2-iminobiotin, avidin, streptavidin, neutravidin. In some embodiments, the binding pair are avidin and streptavidin. In some embodiments, the length of the complementary portion between the additional portion of the detection oligonucleotide comprising a stem-loop portion and the detection probe is, is about, is at least, or is not more than, 8, 9, 10, 11, 12, 13, 14, 15, 16, 17, 18, 19, 20, 21, 22, 23, 24, 25, 30, 31, 32, 33, 34, 35, 36, 37, 38, 39 or 40 nucleotides long, or a range defined by any two of the aforementioned values, for example, 8-40, 8-25, 10-18, 15-25, or 20-40 nucleotides long. In some embodiments, the length of the complementary portion between the additional portion of the detection oligonucleotide comprising a stem-loop portion and the detection probe is 20 nucleotides long or about 20 nucleotides long. In some embodiments, the length of the detection probe and/or the detection sequence is, is about, is at least, or is not more than, 8, 9, 10, 11, 12, 13, 14, 15, 16, 17, 18, 19, 20, 21, 22, 23, 24, 25, 30, 31, 32, 33, 34, 35, 36, 37, 38, 39 or 40 nucleotides long, or a range defined by any two of the aforementioned values, for example, 8-40, 8-25, 10-18, 15-25, or 20-40 nucleotides long. In some embodiments, the detection probe and/or the detection sequence is 20 nucleotides long or about 20 nucleotides long. In some embodiments, the complementary portion between the detection sequence of the detection oligonucleotide and the detection probe is fully complementary as measured over the entirety of the portion, optionally as measured over the entirety of the detection probe and/or detection sequence. In some embodiments, the complementary portion between the detection sequence of the detection oligonucleotide and the detection probe as measured over the entirety of the portion, optionally as measured over the entirety of the detection probe and/or detection sequence, is not fully complementary; optionally it is, is about, or is at least, 85%, 86%, 87%, 88%, 89%, 90%, 91%, 92%, 93%, 94%, 95%, 96%, 97%, 98%, or 99% complementary. In some embodiments, the detection oligonucleotides comprising a stem-loop portions is 61-106 nucleotides long, the stem-loop portion is 8-30 nucleotides long, the first portion is 8-24 nucleotides long, the first portion is at least 80% complementary to the capture oligonucleotide as measured over the entire length of the first portion, the second portion is 21-25 nucleotides long, the nucleic acid analyte is 21-25 nucleotides long, the second portion is at least 80% complementary to the nucleic acid analyte as measured over the entire length of the nucleic acid analyte, the detection sequence and the detection probe are each 8-25 nucleotides long, and the detection sequence is at least 80% complementary to the detection probe as measured over the entire length of the detection sequence and/or detection probe. In some embodiments, the detection oligonucleotides comprising a stem-loop portions is 61-106 nucleotides long, the stem-loop portion is 8-30 nucleotides long, the first portion is 8-24 nucleotides long, the first portion is at least 98% complementary to the capture oligonucleotide as measured over the entire length of the first portion, the second portion is 21-25 nucleotides long, the nucleic acid analyte is 21-25 nucleotides long, the second portion is at least 98% complementary to the nucleic acid analyte as measured over the entire length of the nucleic acid analyte, the detection sequence and the detection probe are each 8-25 nucleotides long, and the detection sequence is at least 98% complementary to the detection probe as measured over the entire length of the detection sequence and/or detection probe.

Methods of Detecting an Analyte Using Detection Oligonucleotides with a Stem-Loop Portion and Rolling Circle Amplification

In some embodiments where the detection oligonucleotide comprises a stem-loop structure as described above and elsewhere herein, the detection in item e) comprises the use of an amplification to produce a concatemer attached to the detection oligonucleotide, where the concatemer comprises multiple copies of a detection sequence that is complementary to a sequence in a detection probe. In some embodiments, the detection oligonucleotide having a stem-loop structure comprises a third portion comprising a sequence that is complementary to a sequence in a circular template for use in rolling circle amplification. In some embodiments, the sequence in the detection oligonucleotide that is complementary to a sequence in a circular template for use in rolling circle amplification and the detection sequence that is complementary to a sequence in a detection probe are the same sequence. In some embodiments, they are different sequences. An embodiment of a method using rolling circle amplification is illustrated in FIG. 9. In some embodiments, the method comprises using detection oligonucleotides comprising a stem-loop structures as described above and elsewhere herein, resulting in the detection oligonucleotide having a stem-loop structure being hybridized and/or ligated to an analyte nucleic acid that is ligated to the capture oligonucleotide. The detection oligonucleotide is exposed to a nicking enzyme that cleaves one strand of detection oligonucleotide, thereby nicking the detection oligonucleotide, for example as illustrated in the left side of FIG. 9. This allows the stem-loop portion of the detection oligonucleotide to open. The nicked detection oligonucleotide is exposed to a circular nucleic acid template comprising a sequence that is complementary to the sequence in the third portion of the detection oligonucleotide, and the circular template hybridizes to the detection oligonucleotide as illustrated in middle of FIG. 9. The circular template comprises a detection sequence complementary to a sequence in a detection probe. In some embodiments, the circular template is linear, and is ligated to form a circular template following hybridization to the complementary portion on the detection oligonucleotide, where the hybridization brings the ends of the linear nucleic acid next to each other to permit ligation into a circular nucleic acid. Rolling circle amplification (RCA) is the performed, thereby generating a concatemer attached to the detection oligonucleotide, the concatemer s comprising multiple copies of the detection sequence complementary to the sequence in the detection probe. The concatemer is exposed to detection probes comprising the sequence complementary to the detection sequence, and the detection probes hybridize to the concatemer as illustrated in the right side of FIG. 9, which shows the detection probes as being labeled with an ECL reagent (star). In this way the detection probe hybridizing to the detection oligonucleotide complexes the ECL detection reagent with the detection oligonucleotide (“the complexed ECL detection reagent-detection oligonucleotide”) comprising a concatemer. An ECL reaction is performed to detect hybridized detection probes, optionally after a wash to remove any unhybridized detection probes.

In some embodiments where the detection oligonucleotides have a stem-loop structure comprising a third portion comprising a sequence that is complementary to a sequence in a circular template for use in rolling circle amplification, the detection in item e) comprises: i) exposing the detection oligonucleotides having a stem-loop structure which are hybridized to the ligated nucleic acid analytes and capture oligonucleotides to a nicking enzyme that cleaves one strand of detection oligonucleotides, thereby nicking the detection oligonucleotides; ii) exposing the nicked detection oligonucleotides of i) to circular nucleic acid templates comprising a sequence that is complementary to the sequence in the third portion of the detection oligonucleotides, and which circular templates comprise a detection sequence complementary to a sequence in a detection probe; iii) permitting the circular nucleic acid templates to hybridize to the third portion of the detection oligonucleotides; iv) performing rolling circle amplification (RCA), thereby generating concatemers attached to the detection oligonucleotides, the concatemers comprising multiple copies of the detection sequence complementary to the sequence in the detection probe; v) exposing the concatemers to detection probes comprising the sequence complementary to the detection sequence; vi) permitting hybridization of the detection probes to the concatemers; and vii) conducting the ECL reaction, optionally wherein a wash to remove unhybridized detection probes is performed before conducting the ECL reaction.

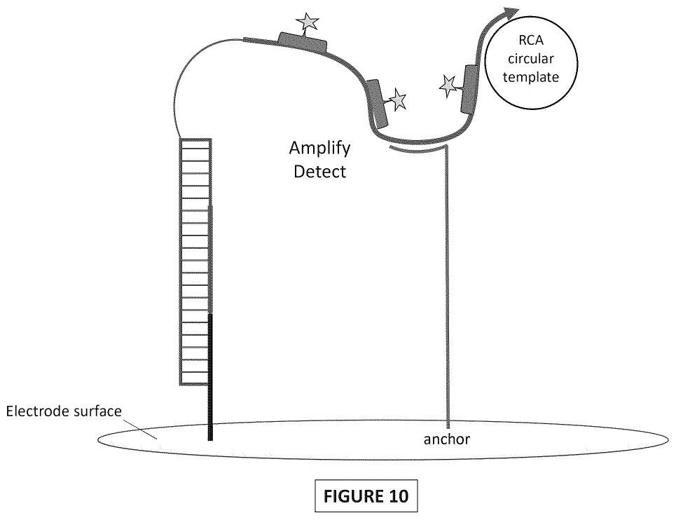

In some embodiments comprising a rolling circle amplification to produce a concatemer attached to the detection oligonucleotide as described above, and elsewhere herein, the method further comprises anchor oligonucleotides immobilized on the surface of the electrode which hybridize to the concatemer to support the concatemer and hold it closer to the surface of the electrode. An illustration of an embodiment comprising an anchor oligonucleotide attached to the surface and hybridized to the concatemer is shown in FIG. 10. In some embodiments, the method further comprises a plurality of anchor oligonucleotides immobilized on the surface of the electrode which are complementary to a sequence in the concatemers, wherein the method comprises hybridizing the anchor oligonucleotides to the concatemers to stabilize the concatemers. In some embodiments, the anchor oligonucleotides are immobilized to the surface of the electrode by hybridization to complementary oligonucleotides that are attached to the surface of the electrode. In some embodiments, the complementary oligonucleotides attached to the surface of the electrode are the capture oligonucleotides. In some embodiments, the anchor oligonucleotides are immobilized to the surface of the electrode by chemical linkage to the electrode surface. In some embodiments, the anchor oligonucleotides are immobilized to the surface of the electrode through a binding pair as described elsewhere herein and according to methods disclosed in international patent application publication numbers WO2020180645 and WO2014160192, each of which is incorporated herein for its disclosure related to immobilization of anchors, and in its entirety.

In some embodiments comprising a rolling circle amplification to produce a concatemer attached to the detection oligonucleotide as described above, and elsewhere herein, the labeling of the detection probe with the ECL detection reagent is by covalent attachment. In some embodiments, the labeling of the detection probe with the ECL detection reagent is through the binding of a binding pair, where the detection probe comprises the first member of the binding pair and the ECL detection reagents comprise the second member of the binding pair. In some embodiments, the detection probe is labeled with the ECL detection reagent prior to hybridization with the detection oligonucleotide. In other embodiments the detection probe is labeled with the ECL detection reagent after it is hybridized to the detection oligonucleotide. In some embodiments, the binding is pair selected from: antibody/antigen, an oligonucleotide/complementary oligonucleotide, and receptor/ligand, optionally selected from biotin, 2-iminobiotin, avidin, streptavidin, neutravidin, glutathione, glutathione s-transferase, maltose, maltose-binding protein, intein, chitin, chitin-binding protein. In some embodiments, the first and second members of the binding pair are selected from biotin, 2-iminobiotin, avidin, streptavidin, neutravidin. In some embodiments, the binding pair are avidin and streptavidin. In some embodiments, the length of the complementary portion between the detection sequence of the detection oligonucleotide and the detection probe is, is about, is at least, or is not more than, 8, 9, 10, 11, 12, 13, 14, 15, 16, 17, 18, 19, 20, 21, 22, 23, 24, 25, 30, 31, 32, 33, 34, 35, 36, 37, 38, 39 or 40 nucleotides long, or a range defined by any two of the aforementioned values, for example, 8-40, 8-25, 10-18, 15-25, or 20-40 nucleotides long. In some embodiments, the complementary portion between the detection sequence of the detection oligonucleotide and the detection probe is 20 nucleotides long or about 20 nucleotides long. In some embodiments, the length of the detection probe and/or the detection sequence is, is about, is at least, or is not more than, 8, 9, 10, 11, 12, 13, 14, 15, 16, 17, 18, 19, 20, 21, 22, 23, 24, 25, 30, 31, 32, 33, 34, 35, 36, 37, 38, 39 or 40 nucleotides long, or a range defined by any two of the aforementioned values, for example, 8-40, 8-25, 10-18, 15-25, or 20-40 nucleotides long. In some embodiments, the detection probe and/or the detection sequence is 20 nucleotides long or about 20 nucleotides long. In some embodiments, the complementary portion between the detection sequence of the detection oligonucleotide and the detection probe is fully complementary as measured over the entirety of the portion, optionally as measured over the entirety of the detection probe and/or the detection sequence. In some embodiments, the complementary portion between the detection sequence of the detection oligonucleotide and the detection probe as measured over the entirety of the portion, optionally as measured over the entirety of the detection probe and/or the detection sequence, is not fully complementary; optionally it is, is about, or is at least, 85%, 86%, 87%, 88%, 89%, 90%, 91%, 92%, 93%, 94%, 95%, 96%, 97%, 98%, or 99% complementary. In some embodiments, the sequence in the third portion of the detection oligonucleotide that is complementary to a sequence in a circular template is the same sequence as the detection sequence that is complementary to a sequence in a detection probe. In some embodiments, they are different sequences. In some embodiments, the length of the complementary portion between the sequence in the detection oligonucleotide and the complementary sequence in a circular template is, is about, is at least, or is not more than, 8, 9, 10, 11, 12, 13, 14, 15, 16, 17, 18, 19, 20, 21, 22, 23, 24, 25, 30, 31, 32, 33, 34, 35, 36, 37, 38, 39 or 40 nucleotides long, or a range defined by any two of the aforementioned values, for example, 8-40, 8-25,10-18, 15-25, or 20-40 nucleotides long. In some embodiments, the complementary portion between the detection oligonucleotide and the circular template is 15 nucleotides long or about 15 nucleotides long. In some embodiments, the complementary portion between the detection oligonucleotide and the circular template is fully complementary as measured over the entirety of the portion. In some embodiments, the complementary portion between the detection oligonucleotide and the circular template as measured over the entirety of the portion, is not fully complementary; optionally it is, is about, or is at least, 85%, 86%, 87%, 88%, 89%, 90%, 91%, 92%, 93%, 94%, 95%, 96%, 97%, 98%, or 99% complementary. In some embodiments, the length of the circular template, is about, is at least, or is not more than, 30, 40, 50, 51, 52, 53, 54, 55, 56, 57, 58, 59, 60, 61, 62, 63, 64, 65, 70, 75, 100, 125, 150, 175, 200, 300, 400, 500, 600, 700, 800, 900, or 1000 nucleotides long, or a range defined by any two of the aforementioned values, for example, 30-1000, 40-1000, 50-150, or 50-100 nucleotides long. In some embodiments, the circular template is 68 nucleotides long or about 68 nucleotides long. In some embodiments, the circular template is provided as a linear nucleic acid and ligated once hybridized to the detection oligonucleotide. In some embodiments, the circular template is provided as a circular nucleic acid. In some embodiments, the detection oligonucleotides comprising a stem-loop portions is 61-106 nucleotides long, the stem-loop portion is 8-30 nucleotides long, the first portion is 8-24 nucleotides long, the first portion is at least 80% complementary to the capture oligonucleotide as measured over the entire length of the first portion, the second portion is 21-25 nucleotides long, the nucleic acid analyte is 21-25 nucleotides long, the second portion is at least 80% complementary to the nucleic acid analyte as measured over the entire length of the nucleic acid analyte, the detection sequence and the detection probe are each 8-25 long, the detection sequence is at least 80% complementary to the detection probe as measured over the entire length of the detection sequence and/or detection probe, the length of the complementary portion between the detection oligonucleotide and the circular template is 8-25 nucleotides long and at least 80% complementary as measured over the entire length of the complementary portion, and the circular template is, is about 40-80 nucleotides long or is, is about 68 nucleotides long. In some embodiments, the detection oligonucleotides comprising a stem-loop portions is 61-106 nucleotides long, the stem-loop portion is 8-30 nucleotides long, the first portion is 8-24 nucleotides long, the first portion is at least 98% complementary to the capture oligonucleotide as measured over the entire length of the first portion, the second portion is 21-25 nucleotides long, the nucleic acid analyte is 21-25 nucleotides long, the second portion is at least 98% complementary to the nucleic acid analyte as measured over the entire length of the nucleic acid analyte, the detection sequence and the detection probe are each 8-25 long, the detection sequence is at least 98% complementary to the detection probe as measured over the entire length of the detection sequence and/or detection probe, the length of the complementary portion between the detection oligonucleotide and the circular template is 8-25 nucleotides long and at least 80% complementary as measured over the entire length of the complementary portion, and the circular template is, is about 40-80 nucleotides long or is, is about 68 nucleotides long.

### Methods Utilizing Multiplexing

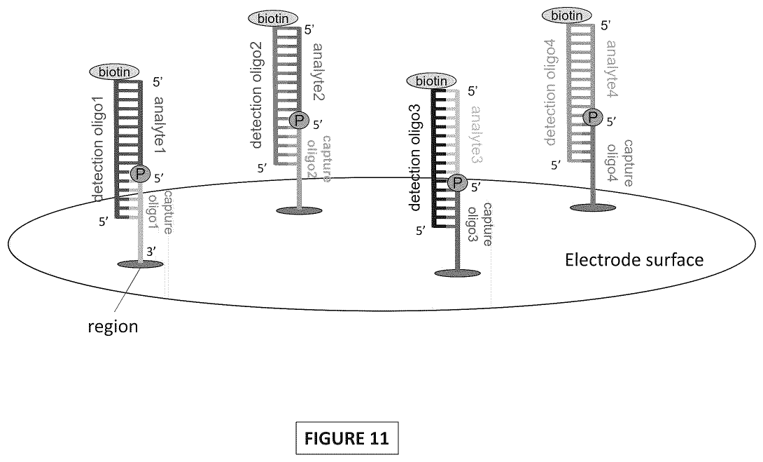

In some embodiments of the methods described above, and elsewhere herein, the method comprises the use of an electrode surface having discrete regions for hybridizing different analytes such that multiple analytes can be detected simultaneously on a single electrode surface by correlating the location of the ECL luminescence with a particular analyte. By placing specific capture oligonucleotides in specific regions of the electrode it will be known which detection oligonucleotides and analytes will hybridize to that region, allowing correlation of luminescence in that region to the presence of the analyte that corresponds to that region (e.g., FIG. 11). In some embodiments, the plurality of detection oligonucleotides provided in item a) comprise a plurality of types of detection oligonucleotides, wherein each type of detection oligonucleotide comprises a first portion and a second portion that corresponds to a capture oligonucleotide sequence and nucleic acid analyte sequence that is different from the other types of detection oligonucleotides. The electrode surface provided in item a) comprises a plurality of regions, each region having a plurality of capture oligonucleotides within the region that hybridize to a single corresponding type of detection oligonucleotide from the plurality of types of detection oligonucleotides, each region hybridizing to a different one of the different types of detection oligonucleotides. The solution of item a) in contact with the electrode surface comprises a plurality of nucleic acid analytes having different nucleic acid sequences, each of which nucleic acid sequences hybridize to a single corresponding type of detection oligonucleotide from the plurality of types of detection oligonucleotides. Item b) of the method comprises i) permitting each of the plurality of different types of detection oligonucleotides to hybridize to the corresponding capture oligonucleotides within a region, wherein each region of the electrode comprises only a single type of detection oligonucleotide hybridized to its corresponding capture oligonucleotide, and ii) permitting each of the plurality of nucleic acid analytes to hybridize to the corresponding type of detection oligonucleotide; thereby forming the plurality of complexes, each complex comprising a nucleic acid analyte, a detection oligonucleotide and a capture oligonucleotide (“the analyte hybridization complex”). In some embodiments, hybridization of the detection oligonucleotides to the corresponding capture oligonucleotides occurs before, with, or after the hybridization of the nucleic acid analytes to the detection oligonucleotides. In some embodiments, each region has a plurality of identical capture oligonucleotides within the region that hybridize to a single corresponding type of detection oligonucleotide. In some embodiments, the capture oligonucleotides within the region that hybridize to a single corresponding type of detection oligonucleotide are not identical, e.g., they comprise one or more sequence differences even though they all hybridize to a single corresponding type of detection oligonucleotides. In some embodiments, all the detection oligonucleotide in a single type of detection oligonucleotide are identical. In some embodiments, all the detection oligonucleotide in a single type of detection oligonucleotide are not identical, e.g., they comprise one or more sequence differences, even though they all hybridize to the same corresponding capture oligonucleotides and the same corresponding nucleic acid analytes. In some embodiments, the nucleic acid analytes that correspond to a single type of detection oligonucleotide are identical. In some embodiments, the nucleic acid analytes that correspond to a single type of detection oligonucleotide are not identical, e.g., they comprise one or more sequence differences, even though they all hybridize to the same single type of detection oligonucleotide.

### Nucleic Acid Characteristics and Reaction Conditions and Methods

The following descriptions of characteristics and conditions can be utilized in the methods described above, and elsewhere herein, in light of the present disclosure, including the examples, figures and claims, and in view of the knowledge of one of skill in the art.

In some embodiments, the detection oligonucleotide is provided at a concentration that is, is about, is at least, or is not more than, 0.1, 0.2, 0.3, 0.4, 0.5, 0.6, 0.7, 0.8, 0.9, 1, 1.25, 1.5, 1.75, 2, 2.25, 2.5, 3, 3.5, 4, 4.5, 5, 5.5, 6, 6.5, or 7 nM, or any concentration within a range defined by any two of the aforementioned concentrations, for example, 0.1-7 nM, 0.1-2 nM, 1-2 nM, or 0.5-5 nM. In some embodiments, the detection oligonucleotide is provided at a concentration of 2 nM or about 2 nM.

In some embodiments, the capture oligonucleotide is phosphorylated at the 5′ end. In some embodiments, the capture oligonucleotide lacks phosphorylation at the 5′ end. In some embodiments, when the capture oligonucleotides lack phosphorylation at the 5′ end, a kinase (e.g., T4 Polynucleotide Kinase) is added to introduce the 5′ phosphate.

In some embodiments, the 5′ phosphate on the capture nucleotides are ligated to the 3′OH on the analyte in the presence of a ligase (e.g., T4 DNA ligase, T4 RNA ligase) and a buffer.

In some embodiments, the method comprises washing the electrode surface one or more times to remove the unhybridized detection oligonucleotides and the analytes that are not ligated to the capture oligonucleotides. In some embodiments, the washing buffer comprises or consists essentially of NaOH in the range of 0.01 to 10 mM, urea in the range of 0.1-7 mM, hydrochloric acid in the range of 0.1-5 mM, propylene glycol in the range of 5-40%, DMSO in the range of 1-20% and water. In some embodiments, the wash involves subjecting the electrode surface to conditions sufficient to remove the unhybridized detection oligonucleotides and the analytes that are not ligated to the capture oligonucleotides. In some embodiments, the wash is performed in the presence of a salt (e.g., sodium hydroxide (NaOH)) at a concentration that is sufficient to remove the unhybridized detection oligonucleotides and the analytes that are not ligated to the capture oligonucleotides. In some embodiments, the electrode surface is contacted with a wash buffer comprising a salt (e.g. NaOH) at a concentration that is, is about, is at least, or is not more than, 0.01, 0.1, 1, 2, 3, 4, 5, 6, 7, 8, 9 or 10 mM, or any concentration within a range defined by any two of the aforementioned concentrations, for example, 0.01 to 10 mM, 0.01 to 5 mM, 1 to 5 mM, or 0.5 to 2 mM. In some embodiments, the concentration of salt (e.g., NaOH) in the wash buffer is 0.5-1.5 mM or about 0.5-1.5 mM. In some embodiments, the wash is performed at a temperature that is appropriate to remove the unhybridized detection oligonucleotides and the analytes that are not ligated to the capture oligonucleotides. In some embodiments, the temperature at which the wash is performed is, is about, is at least, or is not more than, 20, 25, 27, 30, 32, 35, 37, 40, 45, 50, 55, 60, 65, or 70° C., or any temperature within a range defined by any two of the aforementioned concentrations, for example, 20-70° C., 30-65° C., 30-40° C., or 50-70° C. In some embodiments, the wash is performed at a temperature that is 25-40° C. or about 25-40° C. In some embodiments, the wash is performed at a temperature that is 50-65° C. or about 50-65° C. In some embodiments, the wash is performed at a particular concentration (e.g., 5 mM) of NaOH at a particular temperature (e.g., 37° C.). In some embodiments, the wash is performed at a particular concentration (e.g., 5 mM) of NaOH at a particular temperature (e.g., 60° C.). In some embodiments, the wash is performed for a period of time that is, is about, is at least, or is not more than, 0.1, 0.15, 0.3, 0.25, 0.3, 0.35, 0.4, 0.45, 0.5, 0.75 1, 2, 3, or 4 hours, or any number of hours within a range defined by any two of the aforementioned number of hours, for example, 0.1-0.5 hours, 0.5-1 hour, 0.2-3 hours, or 0.1-4 hours. In some embodiments, the period of time the wash is performed is 0.25 hours. In some embodiments, the period of time the wash is performed is 0.5 hours.

Commercially available ECL instruments have demonstrated exceptional performance and they have become widely used for reasons including their excellent sensitivity, dynamic range, precision, and tolerance of complex sample matrices. In some embodiments, the ECL detection reagent is a species that can be induced to emit ECL and includes, for example: i) organometallic compounds where the metal is from, for example, the noble metals of group VIII, including Ru-containing and Os-containing organometallic compounds such as the tris-bipyridyl-ruthenium (RuBpy) moiety and ii) luminol and related compounds. Species that participate with the ECL label in the ECL process are referred to herein as ECL coreactants. In some embodiments the ECL co-reactant is a commonly used co-reactant, for example, a tertiary amine, oxalate, and persulfate for ECL from RuBpy and hydrogen peroxide for ECL from luminol. For more background on ECL, ECL labels, ECL assays and instrumentation for conducting ECL assays see U.S. Pat. Nos. 5,093,268; 5,147,806; 5,324,457; 5,591,581; 5,597,910; 5,641,623; 5,643,713; 5,679,519; 5,705,402; 5,846,485; 5,866,434; 5,786,141; 5,731,147; 6,066,448; 6,136,268; 5,776,672; 5,308,754; 5,240,863; 6,207,369; 6,214,552 and 5,589,136 and Published PCT Nos. WO99/63347; WOOO/03233; WO99/58962; WO99/32662; WO99/14599; WO98/12539; WO97/36931 and WO98/57154, each of which is incorporated herein by reference in its entirety.

In some embodiments, the nucleic acid analyte is a DNA. In some embodiments, the nucleic acid analyte is an RNA. In some embodiments, the RNA is miRNA, siRNA, shRNA, antisense oligonucleotides (ASO) or combinations thereof. In some embodiments, the RNA is miRNA. In some embodiments, the RNA is siRNA. In some embodiments, the RNA is shRNA. In some embodiments, the nucleic acid analytes are extracted from a samples prior to performing the methods of detecting the nucleic acid analytes in the sample. In some embodiments, the nucleic acid analytes are not extracted from the sample prior to performing the methods of detecting the nucleic acid analytes in the sample. In some embodiments, the sample is pretreated before being used in the detection method. Some non-limiting examples of sample pre-treatment include concentration, dilution, or pre-clearing.

Examples of samples that may be analyzed by the methods of the present invention include, but are not limited to food samples (including food extracts, food homogenates, beverages, etc), environmental samples (e.g., soil samples, environmental sludges, collected environmental aerosols, environmental wipes, water filtrates, etc), industrial samples (e.g., starting materials, products or intermediates from an industrial production process), human clinical samples, veterinary samples and other samples of biological origin. Biological samples that may be analyzed include, but are not limited to, feces, mucosal swabs, physiological fluids and/or samples containing suspensions of cells. Specific examples of biological samples include blood, serum, plasma, feces, mucosal swabs, tissue aspirates, tissue homogenates, cell cultures and cell culture supernatants (including cultures of eukaryotic and prokaryotic cells), urine, saliva, sputum, and cerebrospinal fluid. In some embodiments, sample comprises one or more samples. In some embodiments, the sample contains one or more samples comprising one or more nucleic acid analytes to be detected.

In some embodiments, the electrode surface includes one or more carbon-based electrodes. In some embodiments, the electrode is on a support surface comprising a multi-well plate that includes one or more carbon-based electrodes in one or more of the wells. In some embodiments, the electrode includes a carbon ink electrode. In some embodiments, the support surface includes additional electrode surfaces to provide counter or reference electrodes. In some embodiments the support surface comprises an assay cartridge.

### EXAMPLES

### Example 1. MicroRNA Ligation Assay (See, for Example, FIG. 1)

This example describes an assay design that takes advantage of the ability of T4 DNA ligase to link DNA and RNA oligonucleotides, creating a covalent bond between the analyte and the capture oligonucleotide to allow for more stringent wash steps that remove non-specifically binding molecules. The following method was performed:

Sample pre-treatment and hybridization of detection oligo: 500 uL of detection oligo mix was prepared by combining 5 uL biotin-labeled detection oligo (1 uM), 100 uL stock 25×RNAsecure, 50 uL stock 50× proteinase K, and 345 uL MSD Diluent 54. The detection oligo was 34 nt in length. 10 uL of the detection oligo mix was combined with 40 uL of calibrator or sample with analyte per well in a PCR plate and subjected to the following protocol on a thermocycler:
55° C. for 20 minutes (proteinase K treatment step)
98° C. for 2 minutes (denaturization)
65° C. for 1 minutes (50% ramp down) (hybridization)
4° C. hold (3% ramp down)

Binding to ECL plate: 50 uL of MSD N-PLEX Blocker (cat. no. 3-3018-020035) was added to each well of a capture oligo-coated 96-well, 10-spot MSD MULTI-SPOT ECL plate and incubated at 37° C. for 30 minutes with shaking at 705 rpm. Capture oligos had phosphorylated, exposed 5′ ends and a 12 base long complementary sequence to the detection oligo. The plate was washed three times with DPBS (300 uL/well). 50 uL of hybridization product (analyte hybridized to detection oligo) was added to each well of the ECL plate and incubated at 37° C. for 60 minutes with shaking at 705 rpm.

Ligation step: The wells of the ECL plate were washed three times with DPBS (300 uL/well). T4 DNA ligase mix was prepared according to manufacturer's instructions and 50 uL of the ligase mix was added to each well. The plate was incubated at 37° C. for 30 minutes with shaking at 705 rpm.

Stringent wash: The wells of the ECL plate were washed three times with DPBS (300 uL/well). 50 uL NaOH (1 mM) wash solution was added to the wells and incubated at 37° C. for 15 minutes with shaking at 705 rpm.

Add ECL label: The plate was washed three times with DPBS (300 uL/well). 50 uL SULFO-TAG labeled streptavidin (1 ug/mL) was added to each well and incubated at room temperature for 30 minutes with shaking at 705 rpm.

Read plate: The plate was washed three times with DPBS (300 uL/well). 150 uL MSD GOLD™ Read buffer B was added to each well and the plate was immediately read on a MSD® analyzer.

It was observed that using detection oligos with only a 12 base long complementary sequence to the capture oligo, ligation of the analyte to the capture oligo caused the dissociation of the detection oligo from this longer construct to become very difficult. In the absence of the analyte, the detection oligo and the capture oligo were held together by a 12 bases long complementary region having a theoretical melting temperature of around 34° C. After ligation of the analyte to the capture oligo, the detection oligo complementary sequence was 32 bases long and the theoretical melting temperature increased to over 60° C. This method took advantage of the significant difference in Tm's to wash away the detection oligos that were not bound to analyte and were more easily dissociated during wash, releasing the detection oligo from the capture oligo.

### Example 2. Loop Ligation Assay

The loop ligation assay is suitable for detection of both RNA and DNA. The assay can detect miRNA, differentiating between miRNA variants of different lengths as well as 3′ modifications, adenylation of miRNA. The assay can also detect siRNA such as antisense oligonucleotides and can differentiate between isomiRs. The assay employs a stem-loop detection oligonucleotide (see, for example, FIG. 6) and takes advantage of the three dimensional structure of the detection oligo to increase specificity and sensitivity. The stem-loop structure creates a steric pocket for the binding of analyte. Ligation of analyte to “reactive” (phosphorylated) capture oligonucleotides creates long complementary regions between the stems of the structure. Stringent wash steps can selectively remove unbound detection oligonucleotide.

The following method was performed:

Sample lysis/Hybridization of detection oligonucleotide and analyte: 500 ul of 5× detection oligonucleotide mix was prepared by combining 5 uL of 1 uM detection oligonucleotide (biotin-labeled stem-loop), 100 uL 25×RNAsecure, 50 uL 50× proteinase K and 345 ul MSD Diluent 54. The detection oligo was 76 bases long with a 16 loop region and a 14 double stranded portion. 40 uL of sample with 20-mer miRNA analyte was combined with 10 uL of the 5× detection oligonucleotide mix in a 96-well PCR plate (Thermo Scientific catalog no. AB0600L) and subjected to the following protocol run on a thermocycler:

TABLE 1

| Step | Temperature (° C.) | Time (minutes) |
|---|---|---|
| 1 | 50 | 20 |
| 2 | 95 | 10 |
| 3 | 4 | Hold |
| 4 | 98 | 2 |
| 5 | 65 | 1 (ramp down 50%) |
| 6 | 4 | Hold (ramp down 3%) |

Analyte capture: Fifty uL of N-PLEX Blocker (MSD cat. no. 3-3018-020035) was added to each well of a 96-well, 10-spot MSD MULTI-SPOT® plate coated with immobilized capture oligonucleotides phosphorylated at their exposed 5′ ends. The plate was incubated at 37° C. for 30 minutes and then washed three times with 1×DPBS wash buffer. Fifty uL of hybridization product (analyte-detection oligonucleotide complex) was then added to the wells of the ECL plate and the plate was incubated at 37° C. for one hour on a thermoshaker. In this step the analyte-detection oligonucleotide complex hybridized to the capture oligonucleotides immobilized on the plate (“the analyte hybridization complex”).

Ligation: The plate was washed three times with 1×DPBS and 50 uL of T4 DNA ligase mixture (50 mM Tris-HCl, 10 mM DTT, 10 mM MgCl₂, 1 mM ATP, 2.5 mg/ml poly BSA type I, 15 nM circular oligonucleotide template, 0.1 ug/ml T4 ligase) was added to each well. The plate was sealed and incubated at 37° C. with shaking (700 rpm) for 30 minutes.

Wash: The plate was washed 3 times with 1×DPBS. 50 uL per well of stringent wash buffer (1 mM NaOH) was added and the plate was sealed and incubated at 37° C. with shaking (700 rpm) for 15 minutes.

Detection: The plate was washed 3 times with 1×DPBS and 50 uL per well of MSD SULFO-TAG® labeled streptavidin (Meso Scale Discovery, LLC.) was added to each well. The plate was sealed and incubated at 37° C. with shaking (700 rpm) for 30 minutes. The plate was then washed 3 times with 1×DPBS and 150 uL/well of MSD GOLD™ Read Buffer B was added to the plate which was immediately read on an MSD SECTOR® 6000 Imager.

Results/Conclusions: The stem loop detection oligo had a 5′ overhang that was complementary to the capture oligo adjacent to the analyte complementary region, creating a “pocket” hybridization site that was specific for the sequence and also length of the analyte. A biotin modified dTTP was placed in the loop structure to allow for detection. Non-specific signal caused by other RNA molecules with partial complementarity to the hybridization site became theoretically more challenging due to the steric hindrance caused by the flanking DNA. The 5′ phosphate group of the miRNA analyte was able to be ligated to the stem loop structure of the detection oligo in the same ligation reaction that attached the 3′ end of the miRNA analyte to the capture oligo. This created a covalently linked bridge between the capture oligo and the analyte and the detection oligo. This experiment showed promising results with the Loop Ligation Assay in our 20-mer RNA model system.

Table 2 summarizes the results of testing different stringent wash buffers. The estimated lower limit of detection was 20 fM, 12 fM and 9 fM for Hybridization buffer 1, 1 mM NaOH and 4M Urea respectively.

TABLE 2
Test of different stringent wash buffers with 2 nM probe concentration

| | Hybridization buffer 1 | | | | 1 mM NaOH | | | | 4M Urea | | | |
|---|---|---|---|---|---|---|---|---|---|---|---|---|
| analyte | | | % | Sign/ | | | % | Sign/ | | | % | Sign/ |
| cc (pM) | Signal | CV | Recov | backg. | Signal | CV | Recov | backg. | Signal | CV | Recov | backg. |
| 50 | 144763 | 5.4 | 99 | 499 | 195934 | 0.1 | 99 | 823 | 209525 | 8.3 | 101 | 579 |
| 10 | 32627 | 2.0 | 104 | 113 | 47025 | 2.5 | 105 | 198 | 48373 | 2.2 | 102 | 134 |
| 2 | 6877 | 2.6 | 100 | 24 | 10287 | 1.0 | 103 | 43 | 10811 | 3.2 | 98 | 30 |
| 0.4 | 1592 | 2.8 | 92 | 5.5 | 2169 | 6.3 | 89 | 9.1 | 2555 | 5.0 | 94 | 7.1 |
| 0.08 | 656 | 8.0 | 116 | 2.3 | 744 | 2.4 | 103 | 3.1 | 822 | 1.9 | 103 | 2.3 |
| 0.016 | 386 | 5.7 | 102 | 1.3 | 419 | 9.0 | 154 | 1.8 | 559 | 6.5 | 262 | 1.5 |
| 0.0032 | 388 | 3.8 | | 1.3 | 312 | 8.6 | | 1.3 | 217 | 5.2 | | 0.6 |
| 0 | 290 | 8.3 | | 1.0 | 238 | 1.8 | | 1.0 | 362 | 2.9 | | 1.0 |
| eLLOD | | 0.020 | | | 0.012 | | | | 0.009 | | | |
| Hill | 0.972 | | | | 0.947 | | | | 0.922 | | | |
| slope | | | | | | | | | | | | |

Table 3 summarizes the results of specificity testing performed with stem-loop probes designed against different length analytes. Shortening the target analyte by a single base at its 3′ end reduces signals to below 1%. Analytes with a single base overhang at the 3′ ligation site can generate signal up to 5.3% of the signal generated by the probe specific analyte.

TABLE 3

analyte

| | | 20-mer | 19-mer | 18-mer | 17-mer |
|---|---|---|---|---|---|
| probe | 20-mer | 100.0% | 0.3% | 0.2% | 0.2% |
| | 19-mer | 0.5% | 100.0% | 0.9% | 0.4% |
| | 18-mer | 0.6% | 5.3% | 100.0% | 0.6% |
| | 17-mer | 0.7% | 0.7% | 4.6% | 100.0% |

### Example 3. Loop Ligation Assay with Rolling Circle Amplification (RCA)

This assay resembles that of Example 2 but was designed for assays with higher sensitivity needs. The assay uses a stem-loop detection oligonucleotide that comprises a sequence (“3^{rd} portion”) within the loop and a “nicking-enzyme” restriction site on the 3′ end of the sequence (see, for example, FIG. 9). The detection oligonucleotide allows for a rolling cycle amplification step that results in a tenfold increase in sensitivity. The assay set-up is identical to that is Example 2, but lower detection oligonucleotide concentrations can be used.

Sample lysis/Hybridization of detection oligonucleotide and analyte: 500 ul of 5× detection oligonucleotide mix was prepared by combining 5 uL of 1 uM detection oligonucleotide (stem-loop with nick site), 100 uL 25×RNAsecure, 50 uL 50× proteinase K and 345 ul MSD® Diluent 54. The detection oligo was 91 bases long with a 15 loop region and a 22 double stranded portion. Forty uL of sample with 20-mer RNA analyte was combined with 10 uL of the 5× detection oligonucleotide mix in a 96-well PCR plate (Thermo Scientific catalog no. AB0600L) and subjected to the following protocol run on a thermocycler:

TABLE 4

| Step | Temperature (° C.) | Time (minutes) |
|---|---|---|
| 1 | 50 | 20 |
| 2 | 95 | 10 |
| 3 | 4 | Hold |
| 4 | 98 | 2 |
| 5 | 65 | 1 (ramp down 50%) |
| 6 | 4 | Hold (ramp down 3%) |

Analyte capture: Fifty uL of N-PLEX Blocker was added to each well of a 96-well, 10-spot MSD MULTI-SPOT® ECL plate coated with immobilized 36 base capture oligonucleotides phosphorylated at their exposed 5′ ends, and incubated at 37° C. for 30 minutes. The plate was washed three times with 1×DPBS wash buffer. Fifty uL of hybridization product (analyte-detection oligonucleotide complex) was then added to the wells of the ECL plate in duplicates. The plate was incubated at 37° C. for one hour on a thermoshaker. In this step the analyte-detection oligonucleotide complex hybridized to the capture oligonucleotides immobilized on the plate.

Ligation: 50 uL of T4 DNA ligase mixture (50 mM Tris-HCl, 10 mM DTT, 10 mM MgCl₂, 1 mM ATP, 2.5 mg/ml poly BSA type I, 15 nM circular oligonucleotide template, 0.1 ug/ml T4 ligase) was added to each well. The plate was sealed and incubated at 37° C. with shaking (700 rpm) for 30 minutes.

Wash: The plate was washed 3 times with 1×DPBS. 50 uL per well of stringent wash buffer (1 mM NaOH) was added and the plate was sealed and incubated at 37° C. with shaking (700 rpm) for 15 minutes.

Detection: Plate was washed 3 times with 1×DPBS and 50 uL per well of MSD SULFO-TAG labeled streptavidin (Meso Scale Discovery, LLC.) was added. The plate was sealed and incubated at 37° C. with shaking (700 rpm) for 30 minutes. The plate was then washed 3 times with 1×DPBS and 150 uL/well of MSD GOLD Read Buffer B was added to the plate which was immediately read on an MSD SECTOR 6000 Imager.

Following the stringent wash step, a multicomponent reaction is assembled. This mix contains the MSD ligase mix components, a padlock probe that can be ligated over the loop sequence of the probe to form a circle and a restriction enzyme that opens up the loop region of the probe to generate a 3′ end to prime the RCA amplification. In the next step of the assay, we perform a rolling cycle amplification using a DNA polymerase. SULFO-TAG labeled oligonucleotides complementary to the amplification product will generate signal after addition of read buffer.

### Results/Conclusions:

Table 5 summarizes the results of the LLA in combination with rolling circle amplification. Estimated lower level of detection was 3.5 fM. Hill slope in 0.99 and signal to background ratios are high. The addition of the RCA steps increased assay sensitivity 3-4 fold in repeated experiments.

TABLE 5

| | LLA-RCA | | | |
|---|---|---|---|---|
| analyte cc (pM) | Signal | CV | % Recovery | Sign/Back. |
| 50 | 1579241 | 0.1 | 99.2 | 4492.9 |
| 12.5 | 432971 | 2.5 | 103.7 | 1231.8 |
| 3.13 | 105269 | 4.5 | 98.0 | 299.5 |
| 0.78 | 27356 | 7.1 | 99.1 | 77.8 |
| 0.20 | 7051 | 6.4 | 96.7 | 20.1 |
| 0.049 | 2162 | 3.0 | 103.3 | 6.2 |
| 0.012 | 837 | 11.2 | 110.8 | 2.4 |
| 0.0031 | 430 | 3.0 | 78.0 | 1.2 |
| 0.00076 | 355 | 12.0 | | 1.0 |
| 0.00019 | 351 | 2.2 | | 1.0 |
| 0 | 352 | 11.8 | | 1.0 |

| | |
|---|---|
| eLLOD | 0.0035 |
| Hill slope | 0.99 |
| Signal/backgr | 4493 |

What is claimed is:

1. An ECL method of detecting a nucleic acid analyte, the method comprising,
a. contacting an electrode surface comprising a plurality of capture oligonucleotides immobilized on the electrode surface with a solution comprising:
a plurality of nucleic acid analytes; and
a plurality of detection oligonucleotides,
wherein each of the plurality of detection oligonucleotides comprises a first portion and a second portion adjacent to each other,
wherein the first portion comprises a sequence that is complementary to a sequence in the capture oligonucleotide and the second portion comprises a sequence that is complementary to a sequence in the nucleic acid analyte;
b. permitting at least one of the plurality of detection oligonucleotides to hybridize to at least one of the plurality of capture oligonucleotides and at least one of the plurality of nucleic acid analytes, thereby forming a complex, comprising the nucleic acid analyte, a detection oligonucleotide and a capture oligonucleotide (“the analyte hybridization complex”);
c. incubating the analyte hybridization complex from b) under ligation conditions to ligate the nucleic acid analyte to the capture oligonucleotide;
d. washing the electrode surface to remove any detection oligonucleotides that are not hybridized to the capture oligonucleotide and nucleic acid analyte that are ligated together in c); and
e. detecting the detection oligonucleotide hybridized to the ligated nucleic acid analyte and capture oligonucleotide following d), said detecting comprising performing an ECL reaction by inducing a charge at the electrode surface in the presence of an ECL detection reagent complexed with the detection oligonucleotide (“the complexed ECL detection reagent-detection oligonucleotide”) and an ECL co-reactant, and measuring any luminescence signal emitted.

2. The method of claim 1, wherein the detection oligonucleotide comprises a first member of a binding pair and the ECL detection reagents comprises a second member of a binding pair, and wherein binding of the first and second members of the binding pair form the complexed ECL detection reagent-detection oligonucleotide.

3. The method of claim 1, wherein the detection oligonucleotides further comprise a portion comprising a detection sequence that is complementary to a sequence in detection probes, and wherein e) comprises:
i. exposing the detection oligonucleotides hybridized to the ligated nucleic acid analytes and capture oligonucleotides to the detection probes;
ii. permitting hybridization of the detection probes to the detection oligonucleotides; and
iii. conducting the ECL reaction.

4. The method of any one of claims 1-3, wherein the detection oligonucleotides include a stem-loop structure, wherein the stem portion of the detection oligonucleotide comprises a single-stranded portion and a double-stranded-portion when the stem portion is closed, the single-stranded portion comprising the first portion that comprises a sequence that is complementary to the sequence in the capture oligonucleotide and the second portion that comprises a sequence that is complementary to the sequence in the nucleic acid analyte, wherein the double-stranded portion and the first portion flank the second portion.

5. The method of claim 4, wherein during c) the nucleic acid analyte is ligated to the detection oligonucleotide in addition to the capture oligonucleotide.

6. The method of any one of claims 4-5, wherein the first member of the binding pair is located on the stem portion and/or the loop portion of the detection oligonucleotide.

7. The method of any one of claims 4-6, wherein the detection oligonucleotides comprise a third portion comprising:
a) a sequence that is complementary to a sequence in a circular template for use in rolling circle amplification, and/or
b) a detection sequence that is complementary to a sequence in a detection probe, and wherein the detection oligonucleotides further comprise a sequence recognized by a nicking enzyme.

8. The method of claim 7, wherein e) comprises:
i. exposing the detection oligonucleotides hybridized to the ligated nucleic acid analytes and capture oligonucleotides to a nicking enzyme that cleaves one strand of detection oligonucleotides, thereby nicking the detection oligonucleotides;
ii. exposing the nicked detection oligonucleotides of i) to detection probes comprising a sequence complementary to the detection sequence;
iii. permitting hybridization of the detection probes to the nicked detection oligonucleotides; and
iv. conducting the ECL reaction.

9. The method of claim 7, wherein e) comprises:
i. exposing the detection oligonucleotides hybridized to the ligated nucleic acid analytes and capture oligonucleotides to a nicking enzyme that cleaves one strand of detection oligonucleotides, thereby nicking the detection oligonucleotides;
ii. exposing the nicked detection oligonucleotides of i) to circular nucleic acid templates comprising a sequence that is complementary to the sequence in the third portion of the detection oligonucleotides, and which circular templates comprise a detection sequence complementary to a sequence in a detection probe;
iii. permitting the circular nucleic acid templates to hybridize to the third portion of the detection oligonucleotides;
iv. performing rolling circle amplification (RCA), thereby generating concatemers attached to the detection oligonucleotides, the concatemers comprising multiple copies of the detection sequence complementary to the sequence in the detection probe; and
v. exposing the concatemers to detection probes comprising the sequence complementary to the detection sequence;
vi. permitting hybridization of the detection probes to the concatemers; and
vii. conducting the ECL reaction.

10. The method of claim 7 or 9, wherein the sequence that is complementary to the sequence in the circular template for use in rolling circle amplification and the detection sequence that is complementary to the sequence in the detection probe comprise the same sequence.

11. The method of claim 9 or 10, wherein the method further comprises a plurality of anchor oligonucleotides immobilized on the surface of the electrode which are complementary to a sequence in the concatemers, wherein the method comprises hybridizing the anchor oligonucleotides to the concatemers to stabilize the concatemers.

12. The method of claim 11, wherein the anchor oligonucleotide is immobilized to the surface of the electrode by hybridization to a complementary oligonucleotide that is attached to the surface of the electrode, optionally the capture oligonucleotide, or by chemical linkage to the electrode surface.

13. The method of any one of claims 9-12, wherein the circular nucleic acid template is provided in a linear form, hybridized to the third portion of the detection oligonucleotide, and is then ligated to form a circular template prior to amplification in iv).

14. The method of any one of claim 3, or 8-13, wherein the ECL detection reagent comprises the detection probe.

15. The method of claim 14, wherein the ECL detection reagent is combined with the detection probe after the detection probe is hybridized to the detection oligonucleotide or concatemer.

16. The method of any one of the preceding claims, wherein the hybridization of the detection oligonucleotides to the nucleic acid analytes is performed prior to exposing the detection oligonucleotides and the nucleic acid analytes to the capture oligonucleotides.

17. The method of any one of claims 1-15, wherein the hybridization of the detection oligonucleotides to the capture oligonucleotides is performed prior to exposing the detection oligonucleotides and the capture oligonucleotides to the nucleic acid analytes.

18. The method of any one of claims 1-15, wherein the hybridization of the detection oligonucleotides to the capture oligonucleotides is performed together with the hybridization of the detection oligonucleotides to the nucleic acid analytes.

19. The method of any one of the preceding claims, wherein the length of the capture oligonucleotide is, is about, is at least, or is not more than, 8, 9, 10, 11, 12, 13, 14, 15, 16, 17, 18, 19, 20, 21, 22, 23, 24, 25, 30, 35, 36, 37, 38, 39, 40, 41, 42, 43, 44, 45, 46, 47, 48, 49 or 50 nucleotides long, or a range defined by any two of the preceding values, optionally 8-50 nucleotides long, optionally 36 nucleotides long.

20. The method of any one of the preceding claims, wherein the length of the detection oligonucleotide is, is about, is at least, or is not more than, 28, 29, 30, 31, 32, 33, 34, 35, 36, 37, 38, 39, 40, 41, 42, 43, 44, 45, 46, 47, 48, 49, 50, 55, 60, 61, 62, 63, 64, 65, 70, 71, 72, 73, 74, 75, 76, 77, 78, 79, 80, 85, 90, 91, 92, 93, 94, 95, 96, 97, 98, 99, 100, 101, 102, 103, 104, 105 or 106, nucleotides long, or a range defined by any two of the preceding values, optionally 28-40, or 61-91, or 76-106 nucleotides long, optionally 32 nucleotides long, optionally 71 nucleotides long, or optionally 91 nucleotides long.

21. The method of any one of the preceding claims, wherein the nucleic acid analyte is, is about, is at least, or is not more than 16, 17, 18, 19, 20, 21, 22, 23, 24, 25, 30, 31, 32, 33, 34, or 35 nucleotides long, or a range defined by any two of the preceding values, optionally the nucleic acid analyte is 21, 22, 23, 24, or 25 nucleotides long, or a range defined by any two of the preceding values, optionally, 21-23, or 21-25 nucleotides long, optionally 21-23 nucleotides long, optionally 21-25 nucleotides long.

22. The method of any one of the preceding claims, wherein the anchor oligonucleotide is, is about, is at least, or is not more than, 10, 11, 12, 13, 14, 15, 16, 17, 18, 19, 20, 21, 22, 23, 24, 25, 30, 35, 40, 45, 50, 55 or 60 nucleotides long, or a range defined by any two of the preceding values, optionally 24-60 nucleotides long, optionally 49 nucleotides long.

23. The method of any one of the preceding claims, wherein the first portion of the detection oligonucleotide that is complementary to the sequence in the capture oligonucleotides is, is about, is at least, or is not more than, 8, 9, 10, 11, 12, 13, 14, 15, 16, 17, 18, 19, 20, 21, 22, 23, 24, 25, 30, 35, or 40 nucleotides long, or a range defined by any two of the preceding values, optionally 8-24 nucleotides long, optionally 12 nucleotides long.

24. The method of any one of the preceding claims, wherein the length of the second portion of the detection oligonucleotide is 0, 1, 2, 3, 4, or 5 nucleotides shorter or longer than the length or the nucleic acid analyte, or a range defined by any two of the preceding values, optionally 0-5, 1-4, 0-3, 2-4 or 3-5 nucleotides shorter or longer.

25. The method of any one of the preceding claims, wherein the length of the second portion of the detection oligonucleotide is longer than the length of the nucleic acid analyte.

26. The method of any one of the preceding claims, wherein a 3′ end of the capture oligonucleotide is immobilized on the electrode surface.

27. The method of claim 26, wherein the single-stranded portion of the detection oligonucleotide comprising the first portion that is complementary to the sequence in the capture oligonucleotide is located at the 5′ end of the detection oligonucleotide.

28. The method of claim 26, wherein the 5′ end of the capture oligonucleotide is ligated to the 3′ end of the nucleic acid analyte.

29. The method of any one of claims 26-28, wherein the 5′ end of the capture oligonucleotide is phosphorylated prior to ligation in c).

30. The method of claim 29, wherein in the absence of a 5′ end phosphorylation of capture oligonucleotide, an enzyme mediated phosphorylation of the 5′ end of capture oligonucleotide occurs prior to ligation in c).

31. The method of any one of claims 1-25, wherein a 5′ end of the capture oligonucleotide is immobilized on the electrode surface.

32. The method of claim 31, wherein the single-stranded portion of the detection oligonucleotide comprising the first portion that is complementary to the sequence in the capture oligonucleotide is located at the 3′ end of the detection oligonucleotide.

33. The method of claim 31, wherein the 3′ end of the capture oligonucleotide is ligated to the 5′ end of the nucleic acid analyte.

34. The method of any one of the preceding claims, wherein the binding pair is selected from: antibody/antigen, an oligonucleotide/complementary oligonucleotide, and receptor/ligand, optionally selected from biotin, 2-iminobiotin, avidin, streptavidin, neutravidin, glutathione, glutathione s-transferase, maltose, maltose-binding protein, intein, chitin, chitin-binding protein.

35. The method of any one of the preceding claims, wherein the nucleic acid analyte is a DNA or RNA.

36. The method of claim 35, wherein the RNA is selected from: miRNA, siRNA, shRNA or antisense oligonucleotides (ASO).

37. The method of any one of the preceding claims, wherein the detection oligonucleotide is a DNA or RNA.

38. The method of any one of the preceding claims, wherein the capture oligonucleotide is a DNA or RNA.

39. The method of any one of the preceding claims, wherein the nucleic acid analytes are from a sample, optionally wherein the sample comprises blood, plasma, or serum.

40. The method of any one of the preceding claims, wherein the nucleic acid analytes are extracted from blood, plasma, or serum samples.

41. The method of any one of the preceding claims,
wherein the plurality of detection oligonucleotides comprises a plurality of types of detection oligonucleotides, wherein each type of detection oligonucleotide comprises a first portion and a second portion that corresponds to a capture oligonucleotide sequence and nucleic acid analyte sequence that is different from the other types of detection oligonucleotides,
wherein the electrode surface comprises a plurality of regions, each region having a plurality of capture oligonucleotides within the region that hybridize to a single corresponding type of detection oligonucleotide from the plurality of types of detection oligonucleotides, each region hybridizing to a different one of the different types of detection oligonucleotides,
wherein the solution in contact with the electrode surface comprises a plurality of nucleic acid analytes having different nucleic acid sequences, each of which nucleic acid sequences hybridize to a single corresponding type of detection oligonucleotide from the plurality of types of detection oligonucleotides, and
wherein b) comprises permitting each of the plurality of different types of detection oligonucleotides to hybridize to the corresponding capture oligonucleotides within a region, wherein each region of the electrode comprises only a single type of detection oligonucleotide hybridized to its corresponding capture oligonucleotide, and permitting each of the plurality of nucleic acid analytes to hybridize to the corresponding type of detection oligonucleotide, thereby forming a plurality of complexes, each complex comprising a nucleic acid analyte, a detection oligonucleotide and a capture oligonucleotide.

42. The method of any one of the preceding claims, wherein the washing buffer comprises formamide.

43. The method of any one of the preceding claims, wherein the washing buffer comprises sodium hydroxide.

44. The method of any one of the preceding claims, wherein the washing buffer comprises urea.

45. The method of any one of the preceding claims, wherein the hybridization is performed with a buffer comprising sodium hydroxide.

46. The method of claim 45, wherein the concentration of sodium hydroxide in the buffer is, is about, is at least, or is not more than, 0.1, 0.5, 1.0, 1.5, 2.0, 2.5, 3.0, 3.5, 4.0, 4.5, 5.0, 5.5 or-6.0 mM, or a range defined by any two of the preceding values, optionally 0.5-2 mM, optionally 1 mM.

47. The method of any one of the preceding claims, wherein the total assay time is, is about, or is less than 7, 6, 5, 4, 3, 2, 1, 0.5, or 0.25 hours or a range defined by any two of the preceding values, optionally 2-5 hours.
